(19) United States
(12) Patent Application Publication
(10) Pub. No.: US 2025/0163164 A1
(43) Pub. Date: May 22, 2025

(54) ANTI-IL-13RA2 MONOCLONAL ANTIBODIES AND USES THEREOF

(71) Applicant: LANOVA MEDICINES DEVELOPMENT CO., LTD., Shanghai (CN)

(72) Inventors: Runsheng LI, Shanghai (CN); Wentao HUANG, Shanghai (CN); Yifan LI, Shanghai (CN); Zhifang LIU, Shanghai (CN); Jie LUO, Shanghai (CN)

(21) Appl. No.: 18/839,651

(22) PCT Filed: Feb. 15, 2023
(86) PCT No.: PCT/CN2023/076131

§ 371 Date: Aug. 19, 2024

(30) Foreign Application Priority Data
Feb. 17, 2022 (WO) PCT/CN2022/076606

(51) Int. Cl. C07K 16/28; A61K 47/68; A61P 35/00

(52) U.S. Cl. CPC C07K 16/2866; A61K 47/68031; A61K 47/6849; A61P 35/00; C07K 2317/24; C07K 2317/732; C07K 2317/77; C07K 2317/92

(57) ABSTRACT

Provided are antibodies or fragment thereof having binding specificity to the human interleukin 13 receptor subunit alpha 2 (IL-13RA2) protein. These antibodies are capable of binding to IL-13RA2 at high affinity and can mediate antibody-dependent cellular cytotoxicity (ADCC) and effectively induce endocytosis. Also provided are methods and uses for treating cancers and inflammatory diseases.

### BACKGROUND

Interleukin 13 receptor subunit alpha 2 (IL-13RA2, or IL-13Rα2), also known as cluster of differentiation 213A2 (CD213A2), is a membrane bound protein that binds IL13. Unlike the other receptor proteins, IL-13RA2 has a short cytoplasmic domain, and does not appear to function as a signal mediator. This protein is, however, able to regulate the effects of both IL-13 and IL-4, even though it does not bind directly to them.

The IL-13 signaling is regulated via a complex receptor system. In non-hematopoietic cells, IL-13 engages a heterodimeric receptor composed of IL-4Rα and IL-13 Rα1. IL-13 Rα1 binds IL-13 with low affinity. When it forms a complex with IL-4Rα, however, it binds with much higher affinity, inducing the effector functions of IL-13. IL-13RA2 is closely related to IL-13 Rα1.

IL-13RA2 has been found to be over-expressed in a variety of cancers, including pancreatic, ovarian, melanomas, and malignant gliomas. Cells with high IL-13RA2 expression can rapidly and efficiently deplete extracellular IL-13. Likewise, IL-13 responses are enhanced in mice lacking IL13RA2. Therefore, IL-13RA2 can act as a decoy receptor for IL-13 and elicits antagonistic activity against IL-13.

IL-13RA2 exists in both membrane-bound and soluble forms. The human soluble form of IL-13RA2 is generated by enzymatic cleavage of the membrane-bound form by matrix metalloproteinase 8.

Overexpression of IL-13RA2 has also been linked to inflammation. For instance, in patients with nasal polyps (NP), IL-13RA2 may contribute to airway inflammation and aberrant remodeling which are the main pathological features.

### SUMMARY

Anti-IL-13RA2 antibodies are discovered herein that have high binding affinity to the human IL-13RA2 protein and are efficient in mediating antibody-dependent cellular cytotoxicity (ADCC), inducing cytotoxicity, and promoting cellular update. These antibodies, therefore, can be suitably used for treating diseases such as cancer and inflammatory diseases, and serve as targeting carrier of antibody-drug conjugates.

In one embodiment, an antibody or fragment thereof is provided that has specificity to the human IL-13RA2 protein. In some embodiments, the antibody or fragment thereof comprises a heavy chain variable region comprising heavy chain complementarity determining regions CDRH1, CDRH2, and CDRH3 and a light chain variable region light chain comprising complementarity determining regions CDRL1, CDRL2, and CDRL3.

In some embodiments, the CDRH1 comprises the amino acid sequence of SEQ ID NO: 25, the CDRH2 comprises the amino acid sequence of SEQ ID NO: 26, the CDRH3 comprises the amino acid sequence of SEQ ID NO: 27, the CDRLI comprises the amino acid sequence of SEQ ID NO: 28, the CDRL2 comprises the amino acid sequence of SEQ ID NO: 29, and the CDRL3 comprises the amino acid sequence of SEQ ID NO: 30.

In some embodiments, the heavy chain variable region comprises an amino acid sequence selected from the group consisting of SEQ ID NO: 13 and 54-57, and the light chain variable region comprises an amino acid sequence selected from the group consisting of SEQ ID NO: 14 and 58-61. In some embodiments, the heavy chain variable region comprises the amino acid sequence of SEQ ID NO:54, and the light chain variable region comprises the amino acid sequence of SEQ ID NO:58.

In some embodiments, the CDRH1 comprises the amino acid sequence of SEQ ID NO: 31, the CDRH2 comprises the amino acid sequence of SEQ ID NO: 32 or 49, the CDRH3 comprises the amino acid sequence of SEQ ID NO: 33, the CDRLI comprises the amino acid sequence of SEQ ID NO: 34, the CDRL2 comprises the amino acid sequence of SEQ ID NO: 35, and the CDRL3 comprises the amino acid sequence of SEQ ID NO: 36. In some embodiments, the CDRH2 comprises the amino acid sequence of SEQ ID NO: 32.

In some embodiments, the heavy chain variable region comprises an amino acid sequence selected from the group consisting of SEQ ID NO: 11 and 62-65, and the light chain variable region comprises an amino acid sequence selected from the group consisting of SEQ ID NO: 12 and 66-69. In some embodiments, the heavy chain variable region comprises the amino acid sequence of SEQ ID NO:62, and the light chain variable region comprises the amino acid sequence of SEQ ID NO:66.

In some embodiments, the CDRHI comprises the amino acid sequence of SEQ ID NO: 37, the CDRH2 comprises the amino acid sequence of SEQ ID NO: 38 or 50, the CDRH3 comprises the amino acid sequence of SEQ ID NO: 39 or 51, the CDRL1 comprises the amino acid sequence of SEQ ID NO: 40, the CDRL2 comprises the amino acid sequence of SEQ ID NO: 41, and the CDRL3 comprises the amino acid sequence of SEQ ID NO: 42. In some embodiments, the CDRH2 comprises the amino acid sequence of SEQ ID NO: 38, and the CDRH3 comprises the amino acid sequence of SEQ ID NO: 39.

In some embodiments, the heavy chain variable region comprises an amino acid sequence selected from the group consisting of SEQ ID NO: 19 and 70-73, and the light chain variable region comprises an amino acid sequence selected from the group consisting of SEQ ID NO: 20 and 74-77. In some embodiments, the heavy chain variable region comprises the amino acid sequence of SEQ ID NO:70, and the light chain variable region comprises the amino acid sequence of SEQ ID NO:74.

In some embodiments, the CDRH1 comprises the amino acid sequence of SEQ ID NO: 43, the CDRH2 comprises the amino acid sequence of SEQ ID NO: 44 or 52, the CDRH3 comprises the amino acid sequence of SEQ ID NO: 45, the CDRLI comprises the amino acid sequence of SEQ ID NO: 46, the CDRL2 comprises the amino acid sequence of SEQ ID NO: 47, and the CDRL3 comprises the amino acid sequence of SEQ ID NO: 48 or 53. In some embodiments, the CDRH2 comprises the amino acid sequence of SEQ ID NO: 44, and the CDRL3 comprises the amino acid sequence of SEQ ID NO: 48.

In some embodiments, the heavy chain variable region comprises an amino acid sequence selected from the group consisting of SEQ ID NO: 21 and 78-81, and the light chain variable region comprises an amino acid sequence selected from the group consisting of SEQ ID NO: 22 and 82-85. In some embodiments, the heavy chain variable region comprises the amino acid sequence of SEQ ID NO:79, and the light chain variable region comprises the amino acid sequence of SEQ ID NO:83.

Also provided, in some embodiments, are polynucleotides encoding the antibody or fragment, antibody-drug conjugates based on the antibody or fragment, and compositions comprising the antibody or fragment thereof and a pharmaceutically acceptable carrier.

Methods and uses for the treatment of diseases and conditions are also provided. In one embodiment, provided is a method of treating cancer or inflammatory in a patient in need thereof, comprising administering to the patient the antibody or fragment thereof of the present disclosure.

### BRIEF DESCRIPTION OF THE DRAWINGS

FIG. 1A-B show the binding affinity of mouse antibodies to IL-13RA2 on A375 cells.

FIG. 2A-B show the binding affinity of rat antibodies to IL-13RA2 on A375 cells.

FIG. 3 shows the binding affinity of certain chimeric antibodies to IL-13RA2 on A375 cells.

FIG. 4 shows the Antibody-Dependent Cellular Cytotoxicity (ADCC) of the chimeric antibodies.

FIG. 5 shows the cell killing activity of the chimeric antibodies.

FIG. 6A-C show the internationization of the chimeric antibodies.

FIG. 7A-J show the binding affinity of the humanized antibodies.

FIG. 8A-B show the ADCC of the humanized antibodies, in IL-13RA2 overexpression cell lines (A) and Endogenous Gliomas cell lines (B).

FIG. 9A-C show the internationization of selected humanized antibodies.

FIG. 10A-C show the in vitro cytoxities of antibody-drug conjugates using the humanized antibodies.

FIG. 11A-B show the in vivo cytoxities of antibody-drug conjugates using the humanized antibodies.

### DETAILED DESCRIPTION

### Definitions

It is to be noted that the term “a” or “an” entity refers to one or more of that entity; for example, “an antibody,” is understood to represent one or more antibodies. As such, the terms “a” (or “an”), “one or more,” and “at least one” can be used interchangeably herein.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, though preferably less than 25% identity, with one of the sequences of the present disclosure.

A polynucleotide or polynucleotide region (or a polypeptide or polypeptide region) has a certain percentage (for example, 60%, 65%, 70%, 75%, 80%, 85%, 90%, 95%, 98% or 99%) of “sequence identity” to another sequence means that, when aligned, that percentage of bases (or amino acids) are the same in comparing the two sequences.

The term “an equivalent nucleic acid or polynucleotide” refers to a nucleic acid having a nucleotide sequence having a certain degree of homology, or sequence identity, with the nucleotide sequence of the nucleic acid or complement thereof. A homolog of a double stranded nucleic acid is intended to include nucleic acids having a nucleotide sequence which has a certain degree of homology with or with the complement thereof. In one aspect, homologs of nucleic acids are capable of hybridizing to the nucleic acid or complement thereof. Likewise, “an equivalent polypeptide” refers to a polypeptide having a certain degree of homology, or sequence identity, with the amino acid sequence of a reference polypeptide. In some aspects, the sequence identity is at least about 70%, 75%, 80%, 85%, 90%, 95%, 98%, or 99%. In some aspects, the equivalent polypeptide or polynucleotide has one, two, three, four or five addition, deletion, substitution and their combinations thereof as compared to the reference polypeptide or polynucleotide. In some aspects, the equivalent sequence retains the activity (e.g., epitope-binding) or structure (e.g., salt-bridge) of the reference sequence.

As used herein, an “antibody” or “antigen-binding polypeptide” refers to a polypeptide or a polypeptide complex that specifically recognizes and binds to an antigen. An antibody can be a whole antibody and any antigen binding fragment or a single chain thereof. Thus the term “antibody” includes any protein or peptide containing molecule that comprises at least a portion of an immunoglobulin molecule having biological activity of binding to the antigen. Examples of such include, but are not limited to a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein.

The terms “antibody fragment” or “antigen-binding fragment”, as used herein, is a portion of an antibody such as F(ab′)₂, F(ab)₂, Fab′, Fab, Fv, scFv and the like. Regardless of structure, an antibody fragment binds with the same antigen that is recognized by the intact antibody. The term “antibody fragment” includes aptamers, spiegelmers, and diabodies. The term “antibody fragment” also includes any synthetic or genetically engineered protein that acts like an antibody by binding to a specific antigen to form a complex.

A “single-chain variable fragment” or “scFv” refers to a fusion protein of the variable regions of the heavy (VH) and light chains (VL) of immunoglobulins. In some aspects, the regions are connected with a short linker peptide of ten to about 25 amino acids. The linker can be rich in glycine for flexibility, as well as serine or threonine for solubility, and can either connect the N-terminus of the VH with the C-terminus of the VL, or vice versa. This protein retains the specificity of the original immunoglobulin, despite removal of the constant regions and the introduction of the linker. ScFv molecules are known in the art and are described, e.g., in U.S. Pat. No. 5,892,019.

The term antibody encompasses various broad classes of polypeptides that can be distinguished biochemically. Those skilled in the art will appreciate that heavy chains are classified as gamma, mu, alpha, delta, or epsilon (γ, μ, α, δ, ε) with some subclasses among them (e.g., γ1-γ4). It is the nature of this chain that determines the “class” of the antibody as IgG, IgM, IgA IgG, or IgE, respectively. The immunoglobulin subclasses (isotypes) e.g., IgG₁, IgG₂, IgG₃, IgG₄, IgG₅, etc. are well characterized and are known to confer functional specialization. Modified versions of each of these classes and isotypes are readily discernable to the skilled artisan in view of the instant disclosure and, accordingly, are within the scope of the instant disclosure. All immunoglobulin classes are clearly within the scope of the present disclosure, the following discussion will generally be directed to the IgG class of immunoglobulin molecules. With regard to IgG, a standard immunoglobulin molecule comprises two identical light chain polypeptides of molecular weight approximately 23,000 Daltons, and two identical heavy chain polypeptides of molecular weight 53,000-70,000. The four chains are typically joined by disulfide bonds in a “Y” configuration wherein the light chains bracket the heavy chains starting at the mouth of the “Y” and continuing through the variable region.

Antibodies, antigen-binding polypeptides, variants, or derivatives thereof of the disclosure include, but are not limited to, polyclonal, monoclonal, multispecific, human, humanized, primatized, or chimeric antibodies, single chain antibodies, epitope-binding fragments, e.g., Fab, Fab′ and F(ab′)₂, Fd, Fvs, single-chain Fvs (scFv), single-chain antibodies, disulfide-linked Fvs (sdFv), fragments comprising either a VK or VH domain, fragments produced by a Fab expression library, and anti-idiotypic (anti-Id) antibodies (including, e.g., anti-Id antibodies to LIGHT antibodies disclosed herein). Immunoglobulin or antibody molecules of the disclosure can be of any type (e.g., IgG, IgE, IgM, IgD, IgA, and IgY), class (e.g., IgG1, IgG2, IgG3, IgG4, IgA1 and IgA2) or subclass of immunoglobulin molecule.

Light chains are classified as either kappa or lambda (K, λ). Each heavy chain class may be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain.

Both the light and heavy chains are divided into regions of structural and functional homology. The terms “constant” and “variable” are used functionally. In this regard, it will be appreciated that the variable domains of both the light (VK) and heavy (VH) chain portions determine antigen recognition and specificity. Conversely, the constant domains of the light chain (CK) and the heavy chain (CH1, CH2 or CH3) confer important biological properties such as secretion, transplacental mobility, Fc receptor binding, complement binding, and the like. By convention the numbering of the constant region domains increases as they become more distal from the antigen-binding site or amino-terminus of the antibody. The N-terminal portion is a variable region and at the C-terminal portion is a constant region; the CH3 and CK domains actually comprise the carboxy-terminus of the heavy and light chain, respectively.

As indicated above, the variable region allows the antibody to selectively recognize and specifically bind epitopes on antigens. That is, the VK domain and VH domain, or subset of the complementarity determining regions (CDRs), of an antibody combine to form the variable region that defines a three-dimensional antigen-binding site. This quaternary antibody structure forms the antigen-binding site present at the end of each arm of the Y. More specifically, the antigen-binding site is defined by three CDRs on each of the VH and VK chains (i.e. CDR-H1, CDR-H2, CDR-H3, CDR-L1, CDR-L2 and CDR-L3). In some instances, e.g., certain immunoglobulin molecules derived from camelid species or engineered based on camelid immunoglobulins, a complete immunoglobulin molecule may consist of heavy chains only, with no light chains. See, e.g., Hamers-Casterman et al., Nature 363:446-448 (1993).

In naturally occurring antibodies, the six “complementarity determining regions” or “CDRs” present in each antigen-binding domain are short, non-contiguous sequences of amino acids that are specifically positioned to form the antigen-binding domain as the antibody assumes its three dimensional configuration in an aqueous environment. The remainder of the amino acids in the antigen-binding domains, referred to as “framework” regions, show less inter-molecular variability. The framework regions largely adopt a β-sheet conformation and the CDRs form loops which connect, and in some cases form part of, the β -sheet structure. Thus, framework regions act to form a scaffold that provides for positioning the CDRs in correct orientation by inter-chain, non-covalent interactions. The antigen-binding domain formed by the positioned CDRs defines a surface complementary to the epitope on the immunoreactive antigen. This complementary surface promotes the non-covalent binding of the antibody to its cognate epitope. The amino acids comprising the CDRs and the framework regions, respectively, can be readily identified for any given heavy or light chain variable region by one of ordinary skill in the art, since they have been precisely defined (see “Sequences of Proteins of Immunological Interest,” Kabat, E., et al., U.S. Department of Health and Human Services, (1983); and Chothia and Lesk, J. Mol. Biol., 196:901-917 (1987)).

In the case where there are two or more definitions of a term which is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. This particular region has been described by Kabat et al., U.S. Dept. of Health and Human Services, “Sequences of Proteins of Immunological Interest” (1983) and by Chothia et al., J. Mol. Biol. 196:901-917 (1987), which are incorporated herein by reference in their entireties. The CDR definitions according to Kabat and Chothia include overlapping or subsets of amino acid residues when compared against each other. Nevertheless, application of either definition to refer to a CDR of an antibody or variants thereof is intended to be within the scope of the term as defined and used herein. The appropriate amino acid residues which encompass the CDRs as defined by each of the above cited references are set forth in the table below as a comparison. The exact residue numbers which encompass a particular CDR will vary depending on the sequence and size of the CDR. Those skilled in the art can routinely determine which residues comprise a particular CDR given the variable region amino acid sequence of the antibody.

| | Kabat | Chothia |
|---|---|---|
| CDR-H1 | 31-35 | 26-32 |
| CDR-H2 | 50-65 | 52-58 |
| CDR-H3 | 95-102 | 95-102 |
| CDR-L1 | 24-34 | 26-32 |
| CDR-L2 | 50-56 | 50-52 |
| CDR-L3 | 89-97 | 91-96 |

Kabat et al. also defined a numbering system for variable domain sequences that is applicable to any antibody. One of ordinary skill in the art can unambiguously assign this system of “Kabat numbering” to any variable domain sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983).

In addition to table above, the Kabat number system describes the CDR regions as follows: CDR-H1 begins at approximately amino acid 31 (i.e., approximately 9 residues after the first cysteine residue), includes approximately 5-7 amino acids, and ends at the next tryptophan residue. CDR-H2 begins at the fifteenth residue after the end of CDR-H1, includes approximately 16-19 amino acids, and ends at the next arginine or lysine residue. CDR-H3 begins at approximately the thirty third amino acid residue after the end of CDR-H2; includes 3-25 amino acids; and ends at the sequence W-G-X-G, where X is any amino acid. CDR-L1 begins at approximately residue 24 (i.e., following a cysteine residue); includes approximately 10-17 residues; and ends at the next tryptophan residue. CDR-L2 begins at approximately the sixteenth residue after the end of CDR-LI and includes approximately 7 residues. CDR-L3 begins at approximately the thirty third residue after the end of CDR-L2 (i.e., following a cysteine residue); includes approximately 7-11 residues and ends at the sequence F or W-G-X-G, where X is any amino acid.

Antibodies disclosed herein may be from any animal origin including birds and mammals. Preferably, the antibodies are human, murine, donkey, rabbit, goat, guinea pig, camel, llama, horse, or chicken antibodies. In another embodiment, the variable region may be condricthoid in origin (e.g., from sharks).

As used herein, the term “heavy chain constant region” includes amino acid sequences derived from an immunoglobulin heavy chain. A polypeptide comprising a heavy chain constant region comprises at least one of: a CH1 domain, a hinge (e.g., upper, middle, and/or lower hinge region) domain, a CH2 domain, a CH3 domain, or a variant or fragment thereof. For example, an antigen-binding polypeptide for use in the disclosure may comprise a polypeptide chain comprising a CH1 domain; a polypeptide chain comprising a CH1 domain, at least a portion of a hinge domain, and a CH2 domain; a polypeptide chain comprising a CH1 domain and a CH3 domain; a polypeptide chain comprising a CH1 domain, at least a portion of a hinge domain, and a CH3 domain, or a polypeptide chain comprising a CH1 domain, at least a portion of a hinge domain, a CH2 domain, and a CH3 domain. In another embodiment, a polypeptide of the disclosure comprises a polypeptide chain comprising a CH3 domain. Further, an antibody for use in the disclosure may lack at least a portion of a CH2 domain (e.g., all or part of a CH2 domain). As set forth above, it will be understood by one of ordinary skill in the art that the heavy chain constant region may be modified such that they vary in amino acid sequence from the naturally occurring immunoglobulin molecule.

The heavy chain constant region of an antibody disclosed herein may be derived from different immunoglobulin molecules. For example, a heavy chain constant region of a polypeptide may comprise a CH1 domain derived from an IgG₁ molecule and a hinge region derived from an IgG₃ molecule. In another example, a heavy chain constant region can comprise a hinge region derived, in part, from an IgG₁ molecule and, in part, from an IgG₃ molecule. In another example, a heavy chain portion can comprise a chimeric hinge derived, in part, from an IgG₁ molecule and, in part, from an IgG₄ molecule.

As used herein, the term “light chain constant region” includes amino acid sequences derived from antibody light chain. Preferably, the light chain constant region comprises at least one of a constant kappa domain or constant lambda domain.

A “light chain-heavy chain pair” refers to the collection of a light chain and heavy chain that can form a dimer through a disulfide bond between the CL domain of the light chain and the CH1 domain of the heavy chain.

As previously indicated, the subunit structures and three-dimensional configuration of the constant regions of the various immunoglobulin classes are well known. As used herein, the term “VH domain” includes the amino terminal variable domain of an immunoglobulin heavy chain and the term “CH1 domain” includes the first (most amino terminal) constant region domain of an immunoglobulin heavy chain. The CH1 domain is adjacent to the VH domain and is amino terminal to the hinge region of an immunoglobulin heavy chain molecule.

As used herein the term “CH2 domain” includes the portion of a heavy chain molecule that extends, e.g., from about residue 244 to residue 360 of an antibody using conventional numbering schemes (residues 244 to 360, Kabat numbering system; and residues 231-340, EU numbering system; see Kabat et al., U.S. Dept. of Health and Human Services, “Sequences of Proteins of Immunological Interest” (1983). The CH2 domain is unique in that it is not closely paired with another domain. Rather, two N-linked branched carbohydrate chains are interposed between the two CH2 domains of an intact native IgG molecule. It is also well documented that the CH3 domain extends from the CH2 domain to the C-terminal of the IgG molecule and comprises approximately 108 residues.

As used herein, the term “hinge region” includes the portion of a heavy chain molecule that joins the CH1 domain to the CH2 domain. This hinge region comprises approximately 25 residues and is flexible, thus allowing the two N-terminal antigen-binding regions to move independently. Hinge regions can be subdivided into three distinct domains: upper, middle, and lower hinge domains (Roux et al., J. Immunol 161:4083 (1998)).

As used herein the term “disulfide bond” includes the covalent bond formed between two sulfur atoms. The amino acid cysteine comprises a thiol group that can form a disulfide bond or bridge with a second thiol group. In most naturally occurring IgG molecules, the CH1 and CK regions are linked by a disulfide bond and the two heavy chains are linked by two disulfide bonds at positions corresponding to 239 and 242 using the Kabat numbering system (position 226 or 229, EU numbering system).

As used herein, the term “chimeric antibody” will be held to mean any antibody wherein the immunoreactive region or site is obtained or derived from a first species and the constant region (which may be intact, partial or modified in accordance with the instant disclosure) is obtained from a second species. In certain embodiments the target binding region or site will be from a non-human source (e.g. mouse or primate) and the constant region is human.

As used herein, “percent humanization” is calculated by determining the number of framework amino acid differences (i.e., non-CDR difference) between the humanized domain and the germline domain, subtracting that number from the total number of amino acids, and then dividing that by the total number of amino acids and multiplying by 100.

By “specifically binds” or “has specificity to,” it is generally meant that an antibody binds to an epitope via its antigen-binding domain, and that the binding entails some complementarity between the antigen-binding domain and the epitope. According to this definition, an antibody is said to “specifically bind” to an epitope when it binds to that epitope, via its antigen-binding domain more readily than it would bind to a random, unrelated epitope. The term “specificity” is used herein to qualify the relative affinity by which a certain antibody binds to a certain epitope. For example, antibody “A” may be deemed to have a higher specificity for a given epitope than antibody “B,” or antibody “A” may be said to bind to epitope “C” with a higher specificity than it has for related epitope “D.”

As used herein, the terms “treat” or “treatment” refer to both therapeutic treatment and prophylactic or preventative measures, wherein the object is to prevent or slow down (lessen) an undesired physiological change or disorder, such as the progression of cancer. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sport, or pet animals such as dogs, cats, guinea pigs, rabbits, rats, mice, horses, cattle, cows, and so on.

As used herein, phrases such as “to a patient in need of treatment” or “a subject in need of treatment” includes subjects, such as mammalian subjects, that would benefit from administration of an antibody or composition of the present disclosure used, e.g., for detection, for a diagnostic procedure and/or for treatment.

### Anti-IL-13RA2 Antibodies

The present disclosure provides anti-IL-13RA2 antibodies and fragments that have high affinity to the human IL-13RA2 protein, are potent in mediating ADCC and cytotoxicity, and can effectively induce endocytosis. In addition, both in vitro and in vivo testing has demonstrated the efficacies of these antibodies in killing IL-13RA2-expressing cells, and treating cancer. These antibodies, therefore, are suitable agents for treating various diseases featuring overexpressed IL-13RA2, such as cancer and inflammatory diseases.

In accordance with one embodiment of the present disclosure, therefore, provided are antibodies and antigen-binding fragments thereof that are able to bind to IL-13RA2. Example antibodies include those murine ones listed in Table 1 (e.g., 24G6D5, 34A5G9, 42E3C4, 51G2B3, 54A4F7, 57F8H3F1, 61H8C1, 62A8D8, 62F6A8, 72B4H4, 73A11E2 and 74B5E4), as well as humanized ones of Tables 8-11. Also included are those that include the same CDRs as illustrated herein. In some embodiments, the disclosed antibodies and fragments include those that bind to the same epitope as those illustrated here, and those that compete with the instantly disclosed in binding to IL-13RA2.

In accordance with one embodiment of the present disclosure, provided is an antibody or fragment thereof that includes the heavy chain and light chain variable domains with the CDR regions disclosed herein, as well as their biological equivalents.

In one embodiment, the CDRs are those of 61H8C1 or its humanized counterparts, as exemplified in Tables 8B and 8D. In one embodiment, the CDRH1 comprises the amino acid sequence of SEQ ID NO: 25 or a variant thereof having one, two, or three deletions, additions, substitutions or the combinations thereof, the CDRH2 comprises the amino acid sequence of SEQ ID NO: 26 or a variant thereof having one, two, or three deletions, additions, substitutions or the combinations thereof, the CDRH3 comprises the amino acid sequence of SEQ ID NO: 27 or a variant thereof having one, two, or three deletions, additions, substitutions or the combinations thereof, the CDRLI comprises the amino acid sequence of SEQ ID NO: 28 or a variant thereof having one, two, or three deletions, additions, substitutions or the combinations thereof, the CDRL2 comprises the amino acid sequence of SEQ ID NO: 29 or a variant thereof having one, two, or three deletions, additions, substitutions or the combinations thereof, and the CDRL3 comprises the amino acid sequence of SEQ ID NO: 30 or a variant thereof having one, two, or three deletions, additions, substitutions or the combinations thereof.

In one embodiment, the CDRHI comprises the amino acid sequence of SEQ ID NO: 25, the CDRH2 comprises the amino acid sequence of SEQ ID NO: 26, the CDRH3 comprises the amino acid sequence of SEQ ID NO: 27, the CDRL1 comprises the amino acid sequence of SEQ ID NO: 28, the CDRL2 comprises the amino acid sequence of SEQ ID NO: 29, and the CDRL3 comprises the amino acid sequence of SEQ ID NO: 30.

Also provided, in some embodiments, are those that include the same CDRs as 61H8C1 or its humanized counterparts. In some embodiments, the disclosed antibodies and fragments include those that bind to the same epitope as 61H8C1 or its humanized counterparts, and those that compete with any of them in binding to IL-13RA2.

In some embodiments, the heavy chain variable region comprises an amino acid sequence selected from the group consisting of SEQ ID NO:13 (mouse or chimeric) and 54-57 (humanized), or a peptide having at least 90% sequence identity to an amino acid sequence selected from the group consisting of SEQ ID NO:13 (mouse or chimeric) and 54-57 (humanized).

In some embodiments, the light chain variable region comprises an amino acid sequence selected from the group consisting of SEQ ID NO:14 (mouse or chimeric) and 58-61 (humanized), or a peptide having at least 90% sequence identity to an amino acid sequence selected from the group consisting of SEQ ID NO: 14 (mouse or chimeric) and 58-61 (humanized).

In some embodiments, the heavy chain variable region comprises the amino acid sequence of SEQ ID NO:54 and the light chain variable region comprises the amino acid sequence of any one of SEQ ID NO: 58-61. In some embodiments, the heavy chain variable region comprises the amino acid sequence of any one of SEQ ID NO: 54-57 and the light chain variable region comprises the amino acid sequence of SEQ ID NO: 58. In some embodiments, the heavy chain variable region comprises the amino acid sequence of SEQ ID NO: 54 and the light chain variable region comprises the amino acid sequence of SEQ ID NO: 58.

In one embodiment, the CDRs are those of 57F8H3F1 or its humanized counterparts, as exemplified in Tables 9B and 9D. In one embodiment, the CDRHI comprises the amino acid sequence of SEQ ID NO: 31 or a variant thereof having one, two, or three deletions, additions, substitutions or the combinations thereof, the CDRH2 comprises the amino acid sequence of SEQ ID NO: 32 or 49 or a variant thereof having one, two, or three deletions, additions, substitutions or the combinations thereof, the CDRH3 comprises the amino acid sequence of SEQ ID NO: 33 or a variant thereof having one, two, or three deletions, additions, substitutions or the combinations thereof, the CDRLI comprises the amino acid sequence of SEQ ID NO: 34 or a variant thereof having one, two, or three deletions, additions, substitutions or the combinations thereof, the CDRL2 comprises the amino acid sequence of SEQ ID NO: 35 or a variant thereof having one, two, or three deletions, additions, substitutions or the combinations thereof, and the CDRL3 comprises the amino acid sequence of SEQ ID NO: 36 or a variant thereof having one, two, or three deletions, additions, substitutions or the combinations thereof.

In one embodiment, the CDRH1 comprises the amino acid sequence of SEQ ID NO: 31, the CDRH2 comprises the amino acid sequence of SEQ ID NO: 32 or 49, the CDRH3 comprises the amino acid sequence of SEQ ID NO: 33, the CDRLI comprises the amino acid sequence of SEQ ID NO: 34, the CDRL2 comprises the amino acid sequence of SEQ ID NO: 35, and the CDRL3 comprises the amino acid sequence of SEQ ID NO: 36. In one embodiment, the CDRH1 comprises the amino acid sequence of SEQ ID NO: 31, the CDRH2 comprises the amino acid sequence of SEQ ID NO: 32, the CDRH3 comprises the amino acid sequence of SEQ ID NO: 33, the CDRLI comprises the amino acid sequence of SEQ ID NO: 34, the CDRL2 comprises the amino acid sequence of SEQ ID NO: 35, and the CDRL3 comprises the amino acid sequence of SEQ ID NO: 36.

Also provided, in some embodiments, are those that include the same CDRs as 57F8H3F1 or its humanized counterparts. In some embodiments, the disclosed antibodies and fragments include those that bind to the same epitope as 57F8H3F1 or its humanized counterparts, and those that compete with any of them in binding to IL-13RA2.

In some embodiments, the heavy chain variable region comprises an amino acid sequence selected from the group consisting of SEQ ID NO:11 (mouse or chimeric) and 62-65 (humanized), or a peptide having at least 90% sequence identity to an amino acid sequence selected from the group consisting of SEQ ID NO:11 (mouse or chimeric) and 62-65 (humanized).

In some embodiments, the light chain variable region comprises an amino acid sequence selected from the group consisting of SEQ ID NO:12 (mouse or chimeric) and 66-69 (humanized), or a peptide having at least 90% sequence identity to an amino acid sequence selected from the group consisting of SEQ ID NO: 12 (mouse or chimeric) and 66-69 (humanized).

In some embodiments, the heavy chain variable region comprises the amino acid sequence of SEQ ID NO:62 and the light chain variable region comprises the amino acid sequence of any one of SEQ ID NO: 66-69. In some embodiments, the heavy chain variable region comprises the amino acid sequence of any one of SEQ ID NO: 62-65 and the light chain variable region comprises the amino acid sequence of SEQ ID NO: 66. In some embodiments, the heavy chain variable region comprises the amino acid sequence of SEQ ID NO: 62 and the light chain variable region comprises the amino acid sequence of SEQ ID NO: 66.

In one embodiment, the CDRs are those of 72B4H4 or its humanized counterparts, as exemplified in Tables 10B and 10D. In one embodiment, the CDRH1 comprises the amino acid sequence of SEQ ID NO: 37 or a variant thereof having one, two, or three deletions, additions, substitutions or the combinations thereof, the CDRH2 comprises the amino acid sequence of SEQ ID NO: 38 or 50 or a variant thereof having one, two, or three deletions, additions, substitutions or the combinations thereof, the CDRH3 comprises the amino acid sequence of SEQ ID NO: 39 or 51 or a variant thereof having one, two, or three deletions, additions, substitutions or the combinations thereof, the CDRLI comprises the amino acid sequence of SEQ ID NO: 40 or a variant thereof having one, two, or three deletions, additions, substitutions or the combinations thereof, the CDRL2 comprises the amino acid sequence of SEQ ID NO: 41 or a variant thereof having one, two, or three deletions, additions, substitutions or the combinations thereof, and the CDRL3 comprises the amino acid sequence of SEQ ID NO: 42 or a variant thereof having one, two, or three deletions, additions, substitutions or the combinations thereof.

In one embodiment, the CDRHI comprises the amino acid sequence of SEQ ID NO: 37, the CDRH2 comprises the amino acid sequence of SEQ ID NO: 38 or 50, the CDRH3 comprises the amino acid sequence of SEQ ID NO: 39 or 51, the CDRLI comprises the amino acid sequence of SEQ ID NO: 40, the CDRL2 comprises the amino acid sequence of SEQ ID NO: 41, and the CDRL3 comprises the amino acid sequence of SEQ ID NO: 42. In one embodiment, the CDRHI comprises the amino acid sequence of SEQ ID NO: 37, the CDRH2 comprises the amino acid sequence of SEQ ID NO: 38, the CDRH3 comprises the amino acid sequence of SEQ ID NO: 39, the CDRLI comprises the amino acid sequence of SEQ ID NO: 40, the CDRL2 comprises the amino acid sequence of SEQ ID NO: 41, and the CDRL3 comprises the amino acid sequence of SEQ ID NO: 42.

Also provided, in some embodiments, are those that include the same CDRs as 72B4H4 or its humanized counterparts. In some embodiments, the disclosed antibodies and fragments include those that bind to the same epitope as 72B4H4 or its humanized counterparts, and those that compete with any of them in binding to IL-13RA2.

In some embodiments, the heavy chain variable region comprises an amino acid sequence selected from the group consisting of SEQ ID NO:19 (mouse or chimeric) and 70-73 (humanized), or a peptide having at least 90% sequence identity to an amino acid sequence selected from the group consisting of SEQ ID NO: 19 (mouse or chimeric) and 70-73 (humanized).

In some embodiments, the light chain variable region comprises an amino acid sequence selected from the group consisting of SEQ ID NO:20 (mouse or chimeric) and 74-77 (humanized), or a peptide having at least 90% sequence identity to an amino acid sequence selected from the group consisting of SEQ ID NO: 20 (mouse or chimeric) and 74-77 (humanized).

In some embodiments, the heavy chain variable region comprises the amino acid sequence of SEQ ID NO:70 and the light chain variable region comprises the amino acid sequence of any one of SEQ ID NO: 74-77. In some embodiments, the heavy chain variable region comprises the amino acid sequence of any one of SEQ ID NO: 70-73 and the light chain variable region comprises the amino acid sequence of SEQ ID NO: 74. In some embodiments, the heavy chain variable region comprises the amino acid sequence of SEQ ID NO: 70 and the light chain variable region comprises the amino acid sequence of SEQ ID NO: 74.

In one embodiment, the CDRs are those of 73A11E2 or its humanized counterparts, as exemplified in Tables 11B and 11D. In one embodiment, the CDRHI comprises the amino acid sequence of SEQ ID NO: 43 or a variant thereof having one, two, or three deletions, additions, substitutions or the combinations thereof, the CDRH2 comprises the amino acid sequence of SEQ ID NO: 44 or 52 or a variant thereof having one, two, or three deletions, additions, substitutions or the combinations thereof, the CDRH3 comprises the amino acid sequence of SEQ ID NO: 45 or a variant thereof having one, two, or three deletions, additions, substitutions or the combinations thereof, the CDRLI comprises the amino acid sequence of SEQ ID NO: 46 or a variant thereof having one, two, or three deletions, additions, substitutions or the combinations thereof, the CDRL2 comprises the amino acid sequence of SEQ ID NO: 47 or a variant thereof having one, two, or three deletions, additions, substitutions or the combinations thereof, and the CDRL3 comprises the amino acid sequence of SEQ ID NO: 48 or 53 or a variant thereof having one, two, or three deletions, additions, substitutions or the combinations thereof.

In one embodiment, the CDRH1 comprises the amino acid sequence of SEQ ID NO: 43, the CDRH2 comprises the amino acid sequence of SEQ ID NO: 44 or 52, the CDRH3 comprises the amino acid sequence of SEQ ID NO: 45, the CDRLI comprises the amino acid sequence of SEQ ID NO: 46, the CDRL2 comprises the amino acid sequence of SEQ ID NO: 47, and the CDRL3 comprises the amino acid sequence of SEQ ID NO: 48 or 53. In one embodiment, the CDRH1 comprises the amino acid sequence of SEQ ID NO: 43, the CDRH2 comprises the amino acid sequence of SEQ ID NO: 44, the CDRH3 comprises the amino acid sequence of SEQ ID NO: 45, the CDRLI comprises the amino acid sequence of SEQ ID NO: 46, the CDRL2 comprises the amino acid sequence of SEQ ID NO: 47, and the CDRL3 comprises the amino acid sequence of SEQ ID NO: 48.

Also provided, in some embodiments, are those that include the same CDRs as 73A11E2 or its humanized counterparts. In some embodiments, the disclosed antibodies and fragments include those that bind to the same epitope as 73A11E2 or its humanized counterparts, and those that compete with any of them in binding to IL-13RA2.

In some embodiments, the heavy chain variable region comprises an amino acid sequence selected from the group consisting of SEQ ID NO:21 (mouse or chimeric) and 78-81 (humanized), or a peptide having at least 90% sequence identity to an amino acid sequence selected from the group consisting of SEQ ID NO:21 (mouse or chimeric) and 78-81 (humanized).

In some embodiments, the light chain variable region comprises an amino acid sequence selected from the group consisting of SEQ ID NO:22 (mouse or chimeric) and 82-85 (humanized), or a peptide having at least 90% sequence identity to an amino acid sequence selected from the group consisting of SEQ ID NO: 22 (mouse or chimeric) and 82-85 (humanized).

In some embodiments, the heavy chain variable region comprises the amino acid sequence of SEQ ID NO:79 and the light chain variable region comprises the amino acid sequence of any one of SEQ ID NO: 82-85. In some embodiments, the heavy chain variable region comprises the amino acid sequence of any one of SEQ ID NO: 78-81 and the light chain variable region comprises the amino acid sequence of SEQ ID NO: 83. In some embodiments, the heavy chain variable region comprises the amino acid sequence of SEQ ID NO: 79 and the light chain variable region comprises the amino acid sequence of SEQ ID NO: 83.

The antibodies that contained these CDR regions, whether mouse, humanized or chimeric, had potent IL-13RA2 binding activities. As shown in Tables 8-11, certain residues within the CDR can be modified to retain or improve the property or reduce their potential to have post-translational modifications (PTMs). Such modified CDR can be referred to as affinity matured or de-risked CDRs (e.g., SEQ ID NO:49-53).

Modified CDRs can include those having one, two or three amino acid addition, deletion and/or substitutions. In some embodiments, the substitutions can be conservative substitutions.

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in an immunoglobulin polypeptide is preferably replaced with another amino acid residue from the same side chain family. In another embodiment, a string of amino acids can be replaced with a structurally similar string that differs in order and/or composition of side chain family members.

Non-limiting examples of conservative amino acid substitutions are provided in the table below, where a similarity score of 0 or higher indicates conservative substitution between the two amino acids.

TABLE A
Amino Acid Similarity Matrix

| | C | G | P | S | A | T | D | E | N | Q | H | K | R | V | M | I | L | F | Y | W |
|---|---|---|---|---|---|---|---|---|---|---|---|---|---|---|---|---|---|---|---|---|
| W | −8 | −7 | −6 | −2 | −6 | −5 | −7 | −7 | −4 | −5 | −3 | −3 | 2 | −6 | −4 | −5 | −2 | 0 | 0 | 17 |
| Y | 0 | −5 | −5 | −3 | −3 | −3 | −4 | −4 | −2 | −4 | 0 | −4 | −5 | −2 | −2 | −1 | −1 | 7 | 10 | |
| F | −4 | −5 | −5 | −3 | −4 | −3 | −6 | −5 | −4 | −5 | −2 | −5 | −4 | −1 | 0 | 1 | 2 | 9 | | |
| L | −6 | −4 | −3 | −3 | −2 | −2 | −4 | −3 | −3 | −2 | −2 | −3 | −3 | 2 | 4 | 2 | 6 | | | |
| I | −2 | −3 | −2 | −1 | −1 | 0 | −2 | −2 | −2 | −2 | −2 | −2 | −2 | 4 | 2 | 5 | | | | |
| M | −5 | −3 | −2 | −2 | −1 | −1 | −3 | −2 | 0 | −1 | −2 | 0 | 0 | 2 | 6 | | | | | |
| V | −2 | −1 | −1 | −1 | 0 | 0 | −2 | −2 | −2 | −2 | −2 | −2 | −2 | 4 | | | | | | |
| R | −4 | −3 | 0 | 0 | −2 | −1 | −1 | −1 | 0 | 1 | 2 | 3 | 6 | | | | | | | |
| K | −5 | −2 | −1 | 0 | −1 | 0 | 0 | 0 | 1 | 1 | 0 | 5 | | | | | | | | |
| H | −3 | −2 | 0 | −1 | −1 | −1 | 1 | 1 | 2 | 3 | 6 | | | | | | | | | |
| Q | −5 | −1 | 0 | −1 | 0 | −1 | 2 | 2 | 1 | 4 | | | | | | | | | | |
| N | −4 | 0 | −1 | 1 | 0 | 0 | 2 | 1 | 2 | | | | | | | | | | | |
| E | −5 | 0 | −1 | 0 | 0 | 0 | 3 | 4 | | | | | | | | | | | | |
| D | −5 | 1 | −1 | 0 | 0 | 0 | 4 | | | | | | | | | | | | | |
| T | −2 | 0 | 0 | 1 | 1 | 3 | | | | | | | | | | | | | | |
| A | −2 | 1 | 1 | 1 | 2 | | | | | | | | | | | | | | | |
| S | 0 | 1 | 1 | 1 | | | | | | | | | | | | | | | | |
| P | −3 | −1 | 6 | | | | | | | | | | | | | | | | | |
| G | −3 | 5 | | | | | | | | | | | | | | | | | | |
| C | 12 | | | | | | | | | | | | | | | | | | | |

TABLE B
Conservative Amino Acid Substitutions

| For Amino Acid | Substitution With |
|---|---|
| Alanine | D-Ala, Gly, Aib, β-Ala, L-Cys, D-Cys |
| Arginine | D-Arg, Lys, D-Lys, Orn D-Orn |
| Asparagine | D-Asn, Asp, D-Asp, Glu, D-Glu Gln, D-Gln |
| Aspartic Acid | D-Asp, D-Asn, Asn, Glu, D-Glu, Gln, D-Gln |
| Cysteine | D-Cys, S-Me-Cys, Met, D-Met, Thr, D-Thr, L-Ser, |
| | D-Ser |
| Glutamine | D-Gln, Asn, D-Asn, Glu, D-Glu, Asp, D-Asp |
| Glutamic Acid | D-Glu, D-Asp, Asp, Asn, D-Asn, Gln, D-Gln |
| Glycine | Ala, D-Ala, Pro, D-Pro, Aib, β-Ala |
| Isoleucine | D-Ile, Val, D-Val, Leu, D-Leu, Met, D-Met |
| Leucine | Val, D-Val, Met, D-Met, D-Ile, D-Leu, Ile |
| Lysine | D-Lys, Arg, D-Arg, Orn, D-Orn |
| Methionine | D-Met, S-Me-Cys, Ile, D-Ile, Leu, D-Leu, Val, |
| | D-Val |
| Phenylalanine | D-Phe, Tyr, D-Tyr, His, D-His, Trp, D-Trp |
| Proline | D-Pro |
| Serine | D-Ser, Thr, D-Thr, allo-Thr, L-Cys, D-Cys |
| Threonine | D-Thr, Ser, D-Ser, allo-Thr, Met, D-Met, Val, |
| | D-Val |
| Tyrosine | D-Tyr, Phe, D-Phe, His, D-His, Trp, D-Trp |
| Valine | D-Val, Leu, D-Leu, Ile, D-Ile, Met, D-Met |

It will also be understood by one of ordinary skill in the art that antibodies as disclosed herein may be modified such that they vary in amino acid sequence from the naturally occurring binding polypeptide from which they were derived. For example, a polypeptide or amino acid sequence derived from a designated protein may be similar, e.g., have a certain percent identity to the starting sequence, e.g., it may be 60%, 70%, 75%, 80%, 85%, 90%, 95%, 98%, or 99% identical to the starting sequence.

In certain embodiments, the antibody comprises an amino acid sequence or one or more moieties not normally associated with an antibody. Exemplary modifications are described in more detail below. For example, an antibody of the disclosure may comprise a flexible linker sequence, or may be modified to add a functional moiety (e.g., PEG, a drug, a toxin, or a label).

Antibodies, variants, or derivatives thereof of the disclosure include derivatives that are modified, i.e., by the covalent attachment of any type of molecule to the antibody such that covalent attachment does not prevent the antibody from binding to the epitope. For example, but not by way of limitation, the antibodies can be modified, e.g., by glycosylation, acetylation, pegylation, phosphorylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the antibodies may contain one or more non-classical amino acids.

In some embodiments, the antibodies may be conjugated to therapeutic agents, prodrugs, peptides, proteins, enzymes, viruses, lipids, biological response modifiers, pharmaceutical agents, or PEG.

The antibodies may be conjugated or fused to a therapeutic agent, which may include detectable labels such as radioactive labels, an immunomodulator, a hormone, an enzyme, an oligonucleotide, a photoactive therapeutic or diagnostic agent, a cytotoxic agent, which may be a drug or a toxin, an ultrasound enhancing agent, a non-radioactive label, a combination thereof and other such agents known in the art.

The antibodies can be detectably labeled by coupling it to a chemiluminescent compound. The presence of the chemiluminescent-tagged antigen-binding polypeptide is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

The antibodies can also be detectably labeled using fluorescence emitting metals such as ¹⁵²Eu, or others of the lanthanide series. These metals can be attached to the antibody using such metal chelating groups as diethylenetriaminepentacetic acid (DTPA) or ethylenediaminetetraacetic acid (EDTA). Techniques for conjugating various moieties to an antibody are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. (1985); Hellstrom et al., “Antibodies For Drug Delivery”, in Controlled Drug Delivery (2nd Ed.), Robinson et al., (eds.), Marcel Dekker, Inc., pp. 623-53 (1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), Academic Press pp. 303-16 (1985), and Thorpe et al., “The Preparation And Cytotoxic Properties Of Antibody-Toxin Conjugates”, Immunol. Rev. (52:119-58 (1982)).

### Bi-Functional Molecules and Combination Therapies

IL-13RA2 is overexpressed on tumor cells. As a tumor antigen targeting molecule, an antibody or antigen-binding fragment specific to IL-13RA2 can be combined with a second antigen-binding fragment specific to an immune cell, or an antigen-binding fragment specific to an immune checkpoint to generate a combination therapy or a bispecific antibody.

In some embodiments, the immune cell is selected from the group consisting of a T cell, a B cell, a monocyte, a macrophage, a neutrophil, a dendritic cell, a phagocyte, a natural killer cell, an eosinophil, a basophil, and a mast cell. Molecules on the immune cell which can be targeted include, for example, CCL1, CD3, CD16, CD19, CD28, and CD64. Other examples include PD-1,PD-L1, CTLA-4, LAG-3 (also known as CD223), CD28, CD122, 4-1BB (also known as CD137), TIM3, OX-40 or OX40L, CD40 or CD40L, LIGHT, ICOS/ICOSL, GITR/GITRL, TIGIT, CD27, VISTA, B7H3, B7H4, HEVM or BTLA (also known as CD272), killer-cell immunoglobulin-like receptors (KIRs), and CD47.

Different format of bispecific antibodies are also provided. In some embodiments, each of the anti-IL-13RA2 fragment and the second fragment each is independently selected from a Fab fragment, a single-chain variable fragment (scFv), or a single-domain antibody. In some embodiments, the bispecific antibody further includes a Fc fragment.

Bifunctional molecules that include not just antibody or antigen binding fragment are also provided. As a tumor antigen targeting molecule, an antibody or antigen-binding fragment specific to IL-13RA2, such as those described here, can be combined with an immune cytokine or ligand optionally through a peptide linker. The linked immune cytokines or ligands include, but not limited to, IL-2, IL-3, IL-4, IL-5, IL-6, IL-7, IL-10, IL-12, IL-13, IL-15, GM-CSF, TNF-α, CD40L, OX40L, CD27L, CD30L, 4-1BBL, LIGHT and GITRL. Such bi-functional molecules can combine the immune checkpoint blocking effect with tumor site local immune modulation.

### Antibody-Drug Conjugates

In some embodiments, the antibodies or fragments may be conjugated to therapeutic agents, prodrugs, peptides, proteins, enzymes, viruses, lipids, biological response modifiers, pharmaceutical agents, or PEG.

In one embodiment, the antibodies or fragments of the disclosure are covalently attached to a drug moiety. The drug moiety may be, or be modified to include, a group reactive with a conjugation point on the antibody. For example, a drug moiety can be attached by alkylation (e.g., at the epsilon-amino group lysines or the N-terminus of antibodies), reductive amination of oxidized carbohydrate, transesterification between hydroxyl and carboxyl groups, amidation at amino groups or carboxyl groups, and conjugation to thiols.

In some embodiments, the number of drug moieties, p, conjugated per antibody molecule ranges from an average of 1 to 8; 1 to 7, 1 to 6, 1 to 5, 1 to 4, 1 to 3, or 1 to 2. In some embodiments, p ranges from an average of 2 to 8, 2 to 7, 2 to 6, 2 to 5, 2 to 4 or 2 to 3. In other embodiments, p is an average of 1, 2, 3, 4, 5, 6, 7 or 8. In some embodiments, p ranges from an average of about 1 to about 20, about 1 to about 10, about 2 to about 10, about 2 to about 9, about 1 to about 8, about 1 to about 7, about 1 to about 6, about 1 to about 5, about 1 to about 4, about 1 to about 3, or about 1 to about 2. In some embodiments, p ranges from about 2 to about 8, about 2 to about 7, about 2 to about 6, about 2 to about 5, about 2 to about 4 or about 2 to about 3.

For example, when chemical activation of the protein results in formation of free thiol groups, the protein may be conjugated with a sulfhydryl reactive agent. In one aspect, the agent is one which is substantially specific for free thiol groups. Such agents include, for example, malemide, haloacetamides (e.g., iodo, bromo or chloro), haloesters (e.g., iodo, bromo or chloro), halomethyl ketones (e.g., iodo, bromo or chloro), benzylic halides (e.g., iodide, bromide or chloride), vinyl sulfone and pyridylthio.

The drug can be linked to the antibody or fragment by a linker. Suitable linkers include, for example, cleavable and non-cleavable linkers. A cleavable linker is typically susceptible to cleavage under intracellular conditions. Suitable cleavable linkers include, for example, a peptide linker cleavable by an intracellular protease, such as lysosomal protease or an endosomal protease. In exemplary embodiments, the linker can be a dipeptide linker, such as a valine-citrulline (val-cit), a phenylalanine-lysine (phe-lys) linker, or maleimidocapronic-valine-citruline-p-aminobenzyloxycarbonyl (mc-Val-Cit-PABA) linker. Another linker is Sulfosuccinimidyl-4-[N-maleimidomethyl]cyclohexane-1-carboxylate (smcc). Sulfo-smcc conjugation occurs via a maleimide group which reacts with sulfhydryls (thiols, —SH), while its Sulfo-NHS ester is reactive toward primary amines (as found in Lysine and the protein or peptide N-terminus). Yet another linker is maleimidocaproyl (mc). Other suitable linkers include linkers hydrolyzable at a specific pH or a pH range, such as a hydrazone linker. Additional suitable cleavable linkers include disulfide linkers. The linker may be covalently bound to the antibody to such an extent that the antibody must be degraded intracellularly in order for the drug to be released e.g. the mc linker and the like.

A linker can include a group for linkage to the antibody. For example, linker can include an amino, hydroxyl, carboxyl or sulfhydryl reactive groups (e.g., malemide, haloacetamides (e.g., iodo, bromo or chloro), haloesters (e.g., iodo, bromo or chloro), halomethyl ketones (e.g., iodo, bromo or chloro), benzylic halides (e.g., iodide, bromide or chloride), vinyl sulfone and pyridylthio).

In some embodiments, the drug moiety is a cytotoxic or cytostatic agent, an immunosuppressive agent, a radioisotope, a toxin, or the like. The conjugate can be used for inhibiting the multiplication of a tumor cell or cancer cell, causing apoptosis in a tumor or cancer cell, or for treating cancer in a patient. The conjugate can be used accordingly in a variety of settings for the treatment of animal cancers. The conjugate can be used to deliver a drug to a tumor cell or cancer cell. Without being bound by theory, in some embodiments, the conjugate binds to or associates with a cancer cell expressing claudin 18.2, and the conjugate and/or drug can be taken up inside a tumor cell or cancer cell through receptor-mediated endocytosis.

Once inside the cell, one or more specific peptide sequences within the conjugate (e.g., in a linker) are hydrolytically cleaved by one or more tumor-cell or cancer-cell-associated proteases, resulting in release of the drug. The released drug is then free to migrate within the cell and induce cytotoxic or cytostatic or other activities. In some embodiments, the drug is cleaved from the antibody outside the tumor cell or cancer cell, and the drug subsequently penetrates the cell, or acts at the cell surface.

Examples of drug moieties or payloads are selected from the group consisting of DM1 (maytansine, N2′-deacetyl-N2′-(3-mercapto-1-oxopropyl)- or N2′-deacetyl-N2′-(3-mercapto-1-oxopropyl)-maytansine), mc-MMAD (6-maleimidocaproyl-monomethylauristatin-D or N-methyl-L-valyl-N-[(1S,2R)-2-methoxy-4-[(2S)-2-[(1R,2R)-1-methoxy-2-methyl-3-oxo-3-[(1S)-2-phenyl-1-(2-thiazolyl) ethyl]amino]propyl]-1-pyrrolidinyl]-1-[(1S)-1-methylpropyl]-4-oxobutyl]-N-methyl-(9C1)-L-valinamide), mc-MMAF (maleimidocaproyl-monomethylauristatin F or N-[6-(2,5-dihydro-2,5-dioxo-1H-pyrrol-1-yl)-1-oxohexyl]-N-methyl-L-valyl-L-valyl-(3R,4S,5S)-3-methoxy-5-methyl-4-(methylamino) heptanoyl-(αR, βR,2S)-β-methoxy-α-methyl-2-pyrrolidinepropanoyl-L-phenylalanine) and mc-Val-Cit-PABA-MMAE (6-maleimidocaproyl-ValcCit-(p-aminobenzyloxycarbonyl)-monomethylauristatin E or N-[[[4-[N-[6-(2,5-dihydro-2,5-dioxo-1H-pyrrol-1-yl)-1-oxohexyl]-L-valyl-N5-(aminocarbonyl)-L-ornithyl] amino]phenyl] methoxy] carbonyl]-N-methyl-L-valyl-N-[(1S,2R)-4-[(2S)-2-[(1R,2R)-3-[(1R,2S)-2-hydroxy-1-methyl-2-phenylethyl]amino]-1-methoxy-2-methyl-3-oxopropyl]-1-pyrrolidinyl]-2-methoxy-1-[(1S)-1-methylpropyl]-4-oxobutyl]-N-methyl-L-valinamide). DM1 is a derivative of the tubulin inhibitor maytansine while MMAD, MMAE, and MMAF are auristatin derivatives. In some embodiments, the drug moiety is selected from the group consisting of mc-MMAF and mc-Val-Cit-PABA-MMAE. In some embodiments, the drug moiety is a maytansinoid or an auristatin.

The antibodies or fragments may be conjugated or fused to a therapeutic agent, which may include detectable labels such as radioactive labels, an immunomodulator, a hormone, an enzyme, an oligonucleotide, a photoactive therapeutic or diagnostic agent, a cytotoxic agent, which may be a drug or a toxin, an ultrasound enhancing agent, a non-radioactive label, a combination thereof and other such agents known in the art.

The antibodies can be detectably labeled by coupling it to a chemiluminescent compound. The presence of the chemiluminescent-tagged antigen-binding polypeptide is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

The antibodies can also be detectably labeled using fluorescence emitting metals such as ¹⁵²Eu, or others of the lanthanide series. These metals can be attached to the antibody using such metal chelating groups as diethylenetriaminepentacetic acid (DTPA) or ethylenediaminetetraacetic acid (EDTA). Techniques for conjugating various moieties to an antibody are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. (1985); Hellstrom et al., “Antibodies For Drug Delivery”, in Controlled Drug Delivery (2nd Ed.), Robinson et al., (eds.), Marcel Dekker, Inc., pp. 623-53 (1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), Academic Press pp. 303-16 (1985), and Thorpe et al., “The Preparation And Cytotoxic Properties Of Antibody-Toxin Conjugates”, Immunol. Rev. (52:119-58 (1982)).

### Polynucleotides Encoding the Antibodies and Methods of Preparing the Antibodies

The present disclosure also provides isolated polynucleotides or nucleic acid molecules encoding the antibodies, variants or derivatives thereof of the disclosure. The polynucleotides of the present disclosure may encode the entire heavy and light chain variable regions of the antigen-binding polypeptides, variants or derivatives thereof on the same polynucleotide molecule or on separate polynucleotide molecules. Additionally, the polynucleotides of the present disclosure may encode portions of the heavy and light chain variable regions of the antigen-binding polypeptides, variants or derivatives thereof on the same polynucleotide molecule or on separate polynucleotide molecules.

Methods of making antibodies are well known in the art and described herein. In certain embodiments, both the variable and constant regions of the antigen-binding polypeptides of the present disclosure are fully human. Fully human antibodies can be made using techniques described in the art and as described herein. For example, fully human antibodies against a specific antigen can be prepared by administering the antigen to a transgenic animal which has been modified to produce such antibodies in response to antigenic challenge, but whose endogenous loci have been disabled. Exemplary techniques that can be used to make such antibodies are described in U.S. Pat. Nos. 6,150,584; 6,458,592; 6,420,140 which are incorporated by reference in their entireties.

### Treatment Methods

As described herein, the antibodies, variants or derivatives of the present disclosure may be used in certain treatment and diagnostic methods.

The present disclosure is further directed to antibody-based therapies which involve administering the antibodies of the disclosure to a patient such as an animal, a mammal, and a human for treating one or more of the disorders or conditions described herein. Therapeutic compounds of the disclosure include, but are not limited to, antibodies of the disclosure (including variants and derivatives thereof as described herein) and nucleic acids or polynucleotides encoding antibodies of the disclosure (including variants and derivatives thereof as described herein).

The antibodies of the disclosure can also be used to treat or inhibit cancer. In some embodiments, the cancer cells in the patient express or overexpress IL-13RA2. As provided above, IL-13RA2 can be overexpressed in tumor cells, in particular gastric, pancreatic, esophageal, ovarian, and lung tumors. Inhibition of IL-13RA2 has been shown to be useful for treating the tumors.

Accordingly, in some embodiments, provided are methods for treating a cancer in a patient in need thereof. The method, in one embodiment, entails administering to the patient an effective amount of an antibody of the present disclosure. In some embodiments, at least one of the cancer cells (e.g., stromal cells) in the patient over-express IL-13RA2.

Cellular therapies, such as chimeric antigen receptor (CAR) T-cell therapies, are also provided in the present disclosure. A suitable cell can be used, that is put in contact with an anti-IL-13RA2 antibody of the present disclosure (or alternatively engineered to express an anti-IL-13RA2 antibody of the present disclosure). In some embodiments, the antibody is presented in a chimeric antigen receptor (CAR). Upon such contact or engineering, the cell can then be introduced to a cancer patient in need of a treatment. The cancer patient may have a cancer of any of the types as disclosed herein. The cell (e.g., T cell) can be, for instance, a tumor-infiltrating T lymphocyte, a CD4+ T cell, a CD8+ T cell, or the combination thereof, without limitation.

In some embodiments, the cell was isolated from the cancer patient him- or her-self. In some embodiments, the cell was provided by a donor or from a cell bank. When the cell is isolated from the cancer patient, undesired immune reactions can be minimized.

Non-limiting examples of cancers include bladder cancer, breast cancer, colorectal cancer, endometrial cancer, esophageal cancer, head and neck cancer, kidney cancer, leukemia, liver cancer, lung cancer, lymphoma, melanoma, pancreatic cancer, prostate cancer, and thyroid cancer. In some embodiments, the cancer is one or more of gastric, pancreatic, esophageal, ovarian, and lung cancers.

Additional diseases or conditions associated with increased cell survival, that may be treated, prevented, diagnosed and/or prognosed with the antibodies or variants, or derivatives thereof of the disclosure include, but are not limited to, progression, and/or metastases of malignancies and related disorders such as leukemia (including acute leukemias (e.g., acute lymphocytic leukemia, acute myelocytic leukemia (including myeloblastic, promyelocytic, myelomonocytic, monocytic, and erythroleukemia)) and chronic leukemias (e.g., chronic myelocytic (granulocytic) leukemia and chronic lymphocytic leukemia)), polycythemia vera, lymphomas (e.g., Hodgkin's disease and non-Hodgkin's disease), multiple myeloma, Waldenstrom's macroglobulinemia, heavy chain disease, and solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyo sarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma and retinoblastoma.

A specific dosage and treatment regimen for any particular patient will depend upon a variety of factors, including the particular antibodies, variant or derivative thereof used, the patient's age, body weight, general health, sex, and diet, and the time of administration, rate of excretion, drug combination, and the severity of the particular disease being treated. Judgment of such factors by medical caregivers is within the ordinary skill in the art. The amount will also depend on the individual patient to be treated, the route of administration, the type of formulation, the characteristics of the compound used, the severity of the disease, and the desired effect. The amount used can be determined by pharmacological and pharmacokinetic principles well known in the art.

In some embodiments, the antibodies of the present disclosure can be used for treating inflammation or inflammatory diseases. The inflammation, in some embodiments, may be caused by injury, infection, or autoimmune diseases. In some embodiments, the inflammation is a skin inflammation, such as nasal polyps (NP).

Methods of administration of the antibodies, variants or include but are not limited to intradermal, intramuscular, intraperitoneal, intravenous, subcutaneous, intranasal, epidural, and oral routes. The antigen-binding polypeptides or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Thus, pharmaceutical compositions containing the antigen-binding polypeptides of the disclosure may be administered orally, rectally, parenterally, intracistemally, intravaginally, intraperitoneally, topically (as by powders, ointments, drops or transdermal patch), bucally, or as an oral or nasal spray.

The term “parenteral” as used herein refers to modes of administration which include intravenous, intramuscular, intraperitoneal, intrasternal, subcutaneous and intra-articular injection and infusion.

Administration can be systemic or local. In addition, it may be desirable to introduce the antibodies of the disclosure into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

It may be desirable to administer the antigen-binding polypeptides or compositions of the disclosure locally to the area in need of treatment; this may be achieved by, for example, and not by way of limitation, local infusion during surgery, topical application, e.g., in conjunction, with a wound dressing after surgery, by injection, by means of a catheter, by means of a suppository, or by means of an implant, said implant being of a porous, non-porous, or gelatinous material, including membranes, such as sialastic membranes, or fibers. Preferably, when administering a protein, including an antibody, of the disclosure, care must be taken to use materials to which the protein does not absorb.

The amount of the antibodies of the disclosure which will be effective in the treatment, inhibition and prevention of an inflammatory, immune or malignant disease, disorder or condition can be determined by standard clinical techniques. In addition, in vitro assays may optionally be employed to help identify optimal dosage ranges. The precise dose to be employed in the formulation will also depend on the route of administration, and the seriousness of the disease, disorder or condition, and should be decided according to the judgment of the practitioner and each patient's circumstances. Effective doses may be extrapolated from dose-response curves derived from in vitro or animal model test systems.

As a general proposition, the dosage administered to a patient of the antigen-binding polypeptides of the present disclosure is typically 0.1 mg/kg to 100 mg/kg of the patient's body weight, between 0.1 mg/kg and 20 mg/kg of the patient's body weight, or 1 mg/kg to 10 mg/kg of the patient's body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies of the disclosure may be reduced by enhancing uptake and tissue penetration (e.g., into the brain) of the antibodies by modifications such as, for example, lipidation.

In an additional embodiment, the compositions of the disclosure are administered in combination with cytokines. Cytokines that may be administered with the compositions of the disclosure include, but are not limited to, IL-2, IL-3, IL-4, IL-5, IL-6, IL-7, IL-10, IL-12, IL-13, IL-15, anti-CD40, CD40L, and TNF-α.

In additional embodiments, the compositions of the disclosure are administered in combination with other therapeutic or prophylactic regimens, such as, for example, radiation therapy.

### Diagnostic Methods

Over-expression of IL-13RA2 is observed in certain tumor samples, and patients having IL-13RA2-over-expressing cells are likely responsive to treatments with the anti-IL-13RA2 antibodies of the present disclosure. Accordingly, the antibodies of the present disclosure can also be used for diagnostic and prognostic purposes.

A sample that preferably includes a cell can be obtained from a patient, which can be a cancer patient or a patient desiring diagnosis. The cell be a cell of a tumor tissue or a tumor block, a blood sample, a urine sample or any sample from the patient. Upon optional pre-treatment of the sample, the sample can be incubated with an antibody of the present disclosure under conditions allowing the antibody to interact with an IL-13RA2 protein potentially present in the sample. Methods such as ELISA can be used, taking advantage of the anti-IL-13RA2 antibody, to detect the presence of the IL-13RA2 protein in the sample.

Presence of the IL-13RA2 protein in the sample (optionally with the amount or concentration) can be used for diagnosis of cancer, as an indication that the patient is suitable for a treatment with the antibody, or as an indication that the patient has (or has not) responded to a cancer treatment. For a prognostic method, the detection can be done at once, twice or more, at certain stages, upon initiation of a cancer treatment to indicate the progress of the treatment.

### Compositions

The present disclosure also provides pharmaceutical compositions. Such compositions comprise an effective amount of an antibody, and an acceptable carrier. In some embodiments, the composition further includes a second anticancer agent (e.g., an immune checkpoint inhibitor).

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. Further, a “pharmaceutically acceptable carrier” will generally be a non-toxic solid, semisolid or liquid filler, diluent, encapsulating material or formulation auxiliary of any type.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents such as acetates, citrates or phosphates. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose are also envisioned. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences by E. W. Martin, incorporated herein by reference. Such compositions will contain a therapeutically effective amount of the antigen-binding polypeptide, preferably in purified form, together with a suitable amount of carrier so as to provide the form for proper administration to the patient. The formulation should suit the mode of administration. The parental preparation can be enclosed in ampoules, disposable syringes or multiple dose vials made of glass or plastic.

In an embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compounds of the disclosure can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

### EXAMPLES

### Example 1: Generation of Murine and Rat Monoclonal Antibodies Against Human IL-13RA2

The human IL-13RA2 protein was used to immunize different strains of mice and rats, and hybridomas were generated accordingly. IL-13RA2 positive binders were selected and subcloned. Subsequently, in vitro binding and functional screening were carried out and lead antibodies with highest binding affinity and strongest functional potency were identified.

The VH/VL sequences of 12 lead murine antibodies are provided in Table 1 below.

TABLE 1
VH/VL sequence of the lead murine antibodies

| Name | Sequence (CDRs are underlined) | SEQ ID NO: |
|---|---|---|
| 24G6D5 VH | QVQLQQSGPELVKPGTSVKMSCKASGYTFTSNYIHWVKQRPGQGLEWIGW | 1 |
| | IYPGDGSIKYNEKFKGKTTLTADKSSSTVYMLLSSLTSEDSAIYFCAVLR | |
| | RLDYWGQGTTLTVSS | |
| 24G6D5 VL | DIVLTQSPATLSVTPGDSVSLSCRASQSISNNLHWYQQKSHESPRLLIKY | 2 |
| | ASQSISGIPSRFSGSGSGTDFTLSINSVETEDFGMYFCQQSNSWPLTFGA | |
| | GTKLELK | |
| 34A5G9 VH | EVHLQQSGPELVKPGASVKISCKTSGYTFTEYTMHWVKQSHGKSLEWIGR | 3 |
| | INPNNGIAEYKQTFKDKATLAVDKSSSTAYMEFRSLTSEDSAVYYCARGE | |
| | GYGYWFAYWGQGTLVTVSA | |
| 34A5G9 VL | DIQMTQSPASLSASVGETVTITCRTSDNIYSYLAWYQQRQGKSPQLLVSN | 4 |
| | ARTLAEGVPSRFSGSGSGTQFSLKINSLQPEDFGIYYCQHHSGTPFTFGS | |
| | GTNLEIE | |
| 42E3C4 VH | EVKLVESGGGLVRPGGSLKISCAASGFTFSTYGMSWVRQTPEKRLEWVAT | 5 |
| | ISGGGSYTYYPDSVKGRFTISRDNAKNNLYLQMSSLRSEDTALYYCARRD | |
| | LRSAMDYWGQGTSVTVSS | |
| 42E3C4 VL | DIVMTQSQKFMSTSVGDRVSVTCKASQNVDINVAWYQQKPGQSPKALIYS | 6 |
| | ASFRYSGVPDRFTGSGSGTDFTLTISNVQSEDLAEYFCQQYNSYPLTFGG | |
| | GTKLEIK | |
| 51G2B3 VH | QVQLQQSGPELVKPGASVKISCKASGYAFSDSWMNWVKQRPGQGLEWIGR | 7 |
| | IYPGDGDTNYNGKFKGKATLTADKSSNTAYMQLSSLTSVDSAVYFCARSW | |
| | NGSPWFPYWGQGTLVTVSA | |
| 51G2B3 VL | DVLMTQTPLSLPVSLGDQASISCRSSQTIAHRNGNTYLEWYLQKPGQSPK | 8 |
| | LLLYKVSNRFSGVPDRFSGSGSGTDFTLKISRVEAEDLGVYYCFQGSHVT | |
| | WTFGGGTKLEIK | |
| 54A4F7 VH | EVKLVESGGGLVKPGGSLKLSCAASGFTFSSYGMSWVRQTPEKRLEWVAT | 9 |
| | ISGGGSYTYYPDSVKGRFTISRDNAKNNLYLQMSSLRSEDTALYYCARRD | |
| | LRSAMDHWGQGTSVTVSS | |
| 54A4F7 VL | DIVMTQSQKFMSTSVGDRVSVTCRASQNVDINVAWYQQKPGQSPKSLIYS | 10 |
| | ASYRYSGVPDRFTGSGSGTDFTLTISNVQSEDLAEYFCQQYNSYPLTFGG | |
| | GTKLEIR | |
| 57F8H3F1 VH | QVQLQQPGAELVRPGASVKLSCKASGYTFTNYWMNWIKQRPEQGLEWIGR | 11 |
| | IDPYETETHYNQKFRDRAILTVDKSSSTAFMQLSGLTSEDSAVYYCARGY | |
| | GHYYGVDYWGQGTSVTVSS | |
| 57F8H3F1 VL | DIVLTQSPASLAVSLGQRATISCRASENVGNFGISFVNWFQQKPGQPPKL | 12 |
| | LIYAASNLGSGVPARFSGSGSGTDFSLNIHPMEEDDAAMYFCQQSKGVPW | |
| | TFGGGTKLEIK | |
| 61H8C1 VH | QVQLQQSGAELARPGASVMLSCKASGYTFTSYWTQWIKQRPGQGLEGIGA | 13 |
| | IYPGDDDTSYNQKFKGKATLTADKSSSTAYMQLNSLTSEDSAVYYCARGA | |
| | TLLRPFPYWGQGTLVTVSA | |
| 61H8C1 VL | DIVMTQSHKFMSTSVGDRVSITCKASQDVSTAVAWYQQKPGQSPKLLIYS | 14 |
| | ASYRYTGVPDRFTGSGSGTDFTFTISSVQAEDLAVYSCQQHYSIPPTFGG | |
| | GTKLEIK | |
| 62A8D8 VH | EVKLVESGGGLVKPGGSLKLSCAASGFTFSSYGMSWVRQTPEKRLEWVAT | 15 |
| | ISGGGSYTYYPDSVKGRFTISRDNAKNNLYLQMSSLRSEDTALYYCARRD | |
| | LRSAMDYWGQGTSVTVSS | |
| 62A8D8 VL | DIVMTQSQKFMSTSVGDRVSVTCKASQNVDINVAWYQQKPGQSPKALIYS | 16 |
| | ASYRYSGVPDRFTGSGSGTDFTLTISNVQSEDLAEFFCQQYNNYPLTFGG | |
| | GTKLEIK | |
| 62F6A8 VH | QVQIQQSGAELARPGASVMLSCKASGYTFTDYWMQWIKQRPGQGLEGIGA | 17 |
| | IYPGDGDTAYNQKFRGKATLTADKSSSTAYMQLNSLASEDSAVYFCARGA | |
| | TLLRPFPFWGQGTLVTVSA | |
| 62F6A8 VL | DIVMTQSHKFMSTSVGDRVSITCKASQDVSTAVAWYQQKPGQSPKLLIYS | 18 |
| | ASYRYAGVPDRFTGSGSGTDFTFTISSVQAEDLAVYSCQQHYSIPPTFGG | |
| | GTKLEIK | |
| 72B4H4 VH | QVQLQQPGAERVRPGASVKLSCKASGYTFTNYWMNWVKQRPEQGLEWIGR | 19 |
| | IDPYAGETHYNQKFKDKAILTVDKSSSTAYMQLRSLTSEDSAVYYCARGY | |
| | GDSYGMDHWGQGTSVTVSS | |
| 72B4H4 VL | DIVLTQSPASLAVSLGQRATISCRASESVDNYGISFMNWFQQKPGQPPKL | 20 |
| | LIYAASRQGSGDPARFSGSGSGTDFSLNIHPLEEDDTAMYFCQQSKEVPW | |
| | TFGGGTKLEIK | |
| 73A11E2 VH | EVKLVESGGGLVKPGGSLKLSCAASGFTFSSYGMSWVRQTPEKRLEWVAT | 21 |
| | ISGGGSYTYYPDSVKGRFTISRDNARNNLYLQMNSLRSEDTALYYCARRD | |
| | LRSAMDYWGQGTSVTVSS | |
| 73A11E2 VL | DIVMTQSQKFMSTSVGDRVSVTCKASQNVDINVAWYQQKPGQSPKALIYS | 22 |
| | ASYRYSGVPDRFTGSGSGTDFTLTISNVQSEDLAEFFCQQYNSYPLTFGG | |
| | GTKLEIK | |
| 74B5E4 VH | EVKLVESGGGLVKPGGSLRLSCAASGFTFSTYGMSWVRQTPEKRLEWVAT | 23 |
| | ISGGGSYTYYPDSVKGRFTISRDNAKNNLYLQMSSLRSEDTALYYCARRD | |
| | LRSAMDYWGQGTSVTVSS | |
| 74B5E4 VL | DIVMTQSQKFMSTSVGDRVSVTCKASQNVDINVAWYQQKPGQSPKALIYS | 24 |
| | ASYRYSGVPDRFTGSGSGTDFTLTISNVQSEDLAEYFCQQYNTYPLTFGG | |
| | GTKLEIK | |

### Example 2. Binding of Murine Antibodies to IL-13RA2 Protein

This example examined the mouse antibodies, 28 of which, for their ability to bind the human IL-13RA2 protein expressed on A375 cells.

A375 cells that endogenously expressed IL-13RA2 were harvested from flasks. 100 μl of 1×10⁶ cells/ml of cells were incubated with primary antibodies in 4-fold serial dilutions starting from 100 nM to 0.006 nM for 30 minutes on ice. After being washed with 200 μl of FACS buffer twice, cells were incubated with secondary antibody for 30 minutes on ice. Cells were washed with 200 μl of FACS buffer twice and analyzed by FACS.

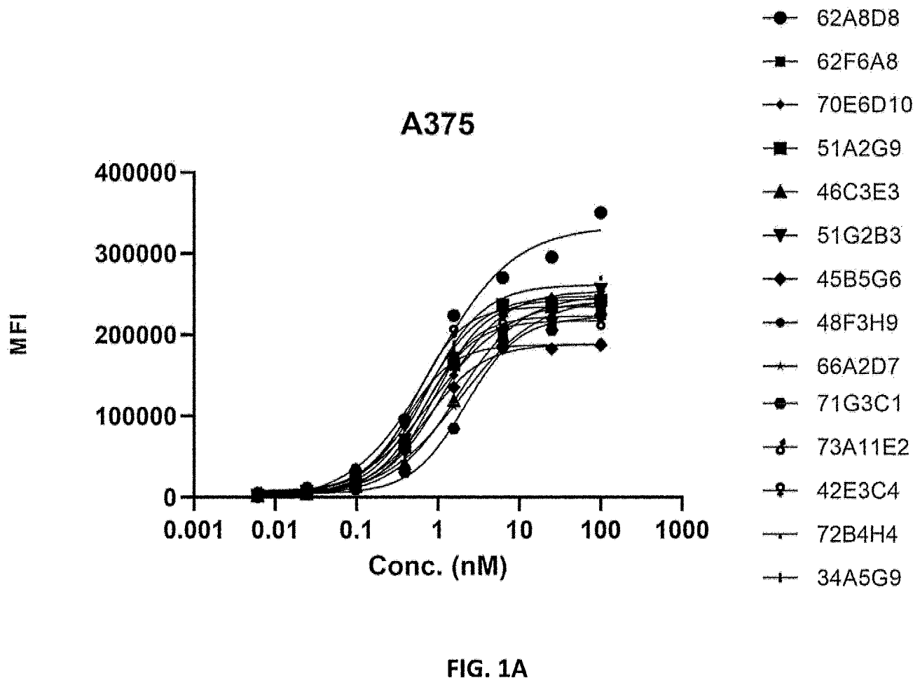

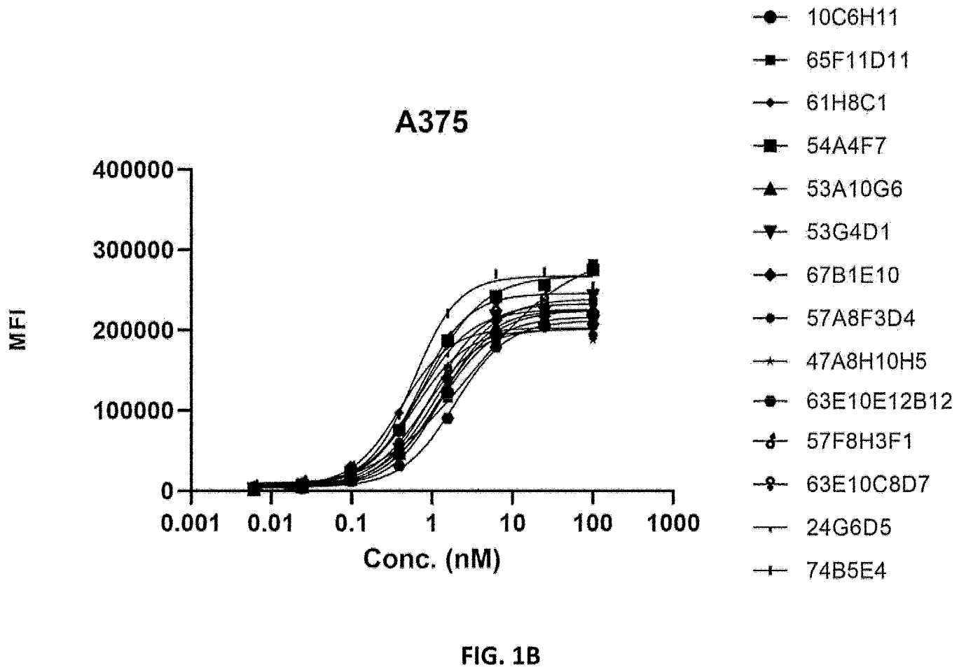

The results of the study (FIG. 1A-B and Table 2) show that the purified murine antibodies could bind to endogenously expressed IL-13RA2 on A375 cells at high EC50.

TABLE 2
Binding Affinity of Purified Mouse Antibodies

| Antibody | EC₅₀ (nM) | Top (MFI) |
|---|---|---|
| 62A8D8 | 0.90 | 333889 |
| 62F6A8 | 0.68 | 217495 |
| 70E6D10 | 1.18 | 254063 |
| 51A2G9 | 0.92 | 244257 |
| 46C3E3 | 1.75 | 249095 |
| 51G2B3 | 0.87 | 242845 |
| 45B5G6 | 0.72 | 188842 |
| 48F3H9 | 0.41 | 187915 |
| 66A2D7 | 1.89 | 246499 |
| 71G3C1 | 2.16 | 222342 |
| 73A11E2 | 0.71 | 223279 |
| 42E3C4 | 0.52 | 235111 |
| 72B4H4 | 0.76 | 261794 |
| 34A5G9 | 0.67 | 248373 |
| 10C6H11 | 1.24 | 216888 |
| 65F11D11 | 3.18 | 302092 |
| 61H8C1 | 0.41 | 202985 |
| 54A4F7 | 0.84 | 267980 |
| 53A10G6 | 1.25 | 224833 |
| 53G4D1 | 1.23 | 239435 |
| 67B1E10 | 1.32 | 225194 |
| 57A8F3D4 | 0.80 | 201907 |
| 47A8H10H5 | 0.60 | 201116 |
| 63E10E12B12 | 1.93 | 212433 |
| 57F8H3F1 | 0.70 | 246001 |
| 63E10C8D7 | 1.00 | 233188 |
| 24G6D5 | 0.68 | 225833 |
| 74B5E4 | 0.60 | 267529 |

### Example 3. Binding of Rat Antibodies to IL-13RA2 Protein

This example examined the rat antibodies, 24 of which, for their ability to bind the human IL-13RA2 protein expressed on A375 cells.

A375 cells that endogenously expressed IL-13RA2 were harvested from flasks. 100 μl of 1×10⁶ cells/ml of cells were incubated with primary antibodies in 4-fold serial dilutions starting from 100 nM to 0.006 nM for 30 minutes on ice. After being washed with 200 μl of FACS buffer twice, cells were incubated with secondary antibody for 30 minutes on ice. Cells were washed with 200 μl of FACS buffer twice and analyzed by FACS.

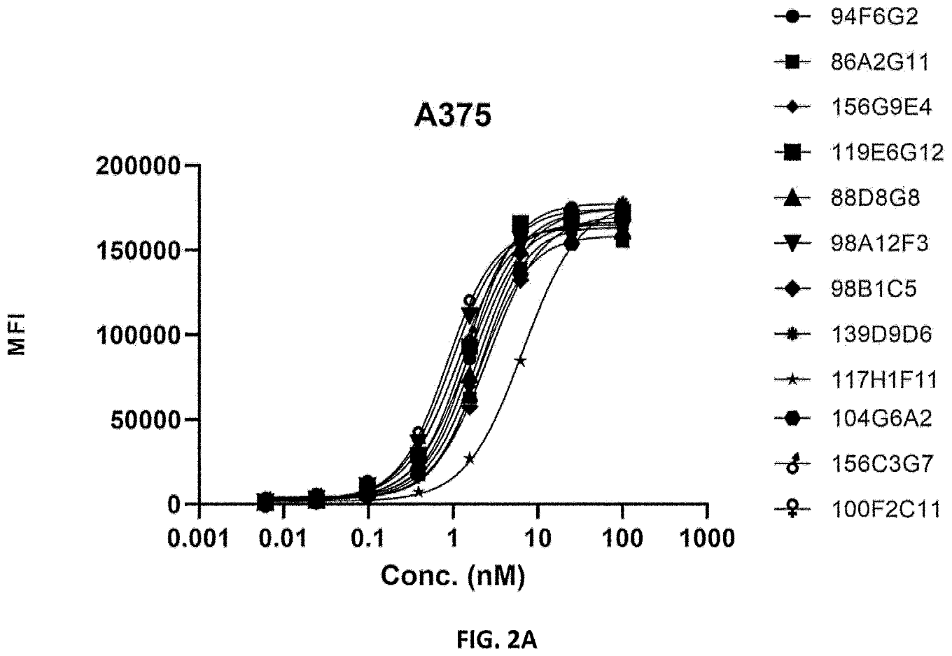

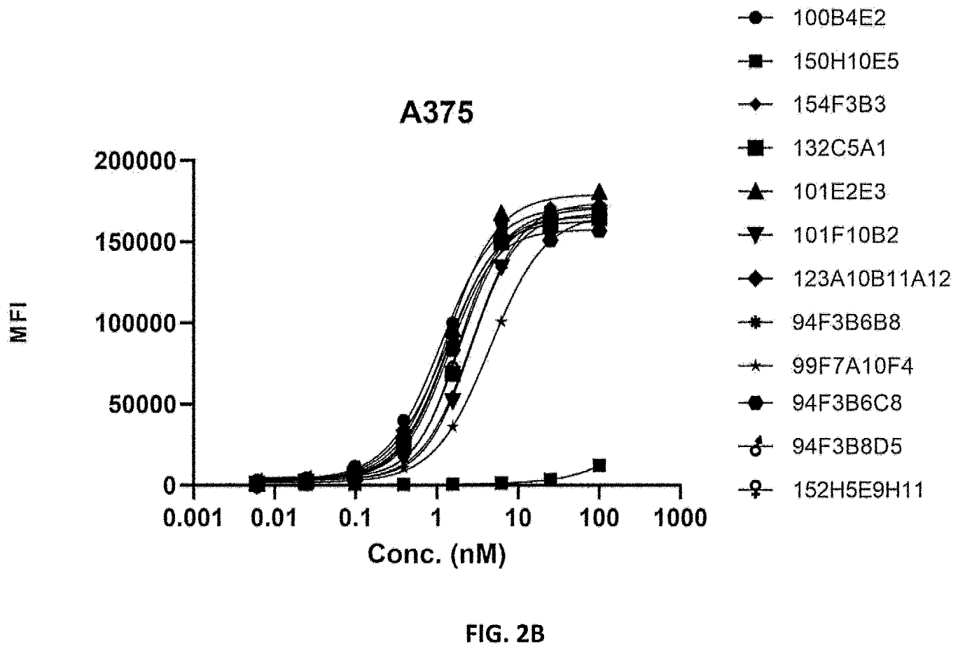

The results of the study (FIG. 2A-B and Table 3) show that the purified rat antibodies could bind to endogenously expressed IL-13RA2 on A375 cells at high EC50.

TABLE 3
Binding Affinity of Purified Rat Antibodies

| Antibody | EC₅₀ (nM) | Top (MFI) |
|---|---|---|
| 94F6G2 | 1.60 | 177701 |
| 86A2G11 | 2.14 | 164044 |
| 156G9E4 | 2.07 | 174584 |
| 119E6G12 | 1.38 | 174436 |
| 88D8G8 | 1.70 | 166609 |
| 98A12F3 | 0.96 | 165026 |
| 98B1C5 | 2.58 | 170793 |
| 139D9D6 | 1.56 | 174468 |
| 117H1F11 | 6.61 | 178554 |
| 104G6A2 | 2.10 | 158706 |
| 156C3G7 | 1.15 | 167075 |
| 100F2C11 | 0.85 | 163131 |
| 100B4E2 | 1.14 | 171789 |
| 150H10E5 | NA | 19998886 |
| 154F3B3 | 2.71 | 174154 |
| 132C5A1 | 1.89 | 165490 |
| 101E2E3 | 1.35 | 179217 |
| 101F10B2 | 2.64 | 167453 |
| 123A10B11A12 | 1.34 | 171246 |
| 94F3B6B8 | 1.32 | 162963 |
| 99F7A10F4 | 4.49 | 167342 |
| 94F3B6C8 | 1.38 | 157537 |
| 94F3B8D5 | 1.36 | 162611 |
| 152H5E9H11 | 1.82 | 165451 |

### Example 4. Binding of Chimeric Antibodies to IL-13RA2 Protein

This example prepared and examined chimeric antibodies (with human Fc) prepared from the murine ones, for their ability to bind the human IL-13RA2 protein expressed on A375 cells.

The murine VH and VK genes were produced synthetically and then respectively cloned into vectors containing the human gamma 1 and human kappa constant domains. The purified chimeric antibodies were produced from transfected CHOs cells.

A375 cells that endogenously expressed IL-13RA2, were harvested from flasks. 100 μl of 1×10⁶ cells/ml of cells were incubated with primary chimeric antibodies in 3-fold serial dilutions starting from 300 nM to 0.002 nM for 30 minutes on ice. After being washed with 200 μl of FACS buffer twice, cells were incubated with secondary antibody for 30 minutes on ice. Cells were washed with 200 μl of FACS buffer twice and analyzed by FACS.

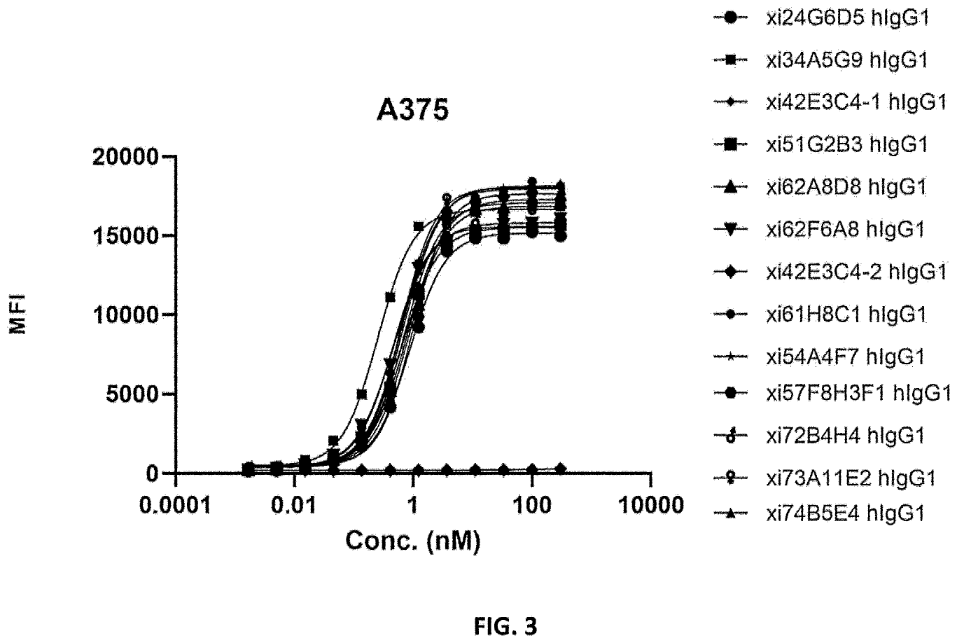

As shown in FIG. 3 and Table 4, the chimeric antibodies bound to human IL-13RA2 at different EC50.

TABLE 4
Binding Affinity of Chimeric Rat Antibodies

| Antibody | EC₅₀ (nM) | Top (MFI) |
|---|---|---|
| xi24G6D5 hIgG1 | 0.88 | 15173 |
| xi34A5G9 hIgG1 | 0.26 | 16673 |
| xi42E3C4-1 hIgG1 | 0.71 | 17081 |
| xi51G2B3 hIgG1 | 0.64 | 15528 |
| xi62A8D8 hIgG1 | 0.82 | 17291 |
| xi62F6A8 hIgG1 | 0.48 | 15815 |
| xi42E3C4-2 hIgG1 | NA | ~351045 |
| xi61H8C1 hIgG1 | 0.47 | 15582 |
| xi54A4F7 hIgG1 | 0.63 | 18048 |
| xi57F8H3F1 hIgG1 | 0.99 | 17646 |
| xi72B4H4 hIgG1 | 0.85 | 16871 |
| xi73A11E2 hIgG1 | 0.65 | 17964 |
| xi74B5E4 hIgG1 | 0.70 | 18142 |

### Example 5. Antibody-Dependent Cellular Cytotoxicity (ADCC) of Chimeric Antibodies

This example measured the antibody-dependent cellular cytotoxicity (ADCC) of the chimeric antibodies.

The ADCC Reporter Bioassay uses an alternative readout at an earlier point in ADCC MOA pathway activation: the activation of gene transcription through the NFAT (nuclear factor of activated T-cells) pathway in the effector cell. In addition, the ADCC Reporter Bioassay uses engineered Jurkat cells stably expressing the FcγRIIIa receptor, V158 (high affinity) variant, and an NFAT response element driving expression of firefly luciferase as effector cells. Antibody biological activity in ADCC MOA is quantified through the luciferase produced as a result of NFAT pathway activation; luciferase activity in the effector cell is quantified with luminescence readout. Signal is high, and assay background is low.

Serial dilutions of IL-13RA2 chimeric monoclonal antibody were incubated for 6 hours of induction at 37° C. with engineered Jurkat effector cells (ADCC Bioassay Effector Cells), with ADCC Bioassay Target Cells (A375 cell line). Luciferase activity was quantified using Bio-Glo™ Reagent.

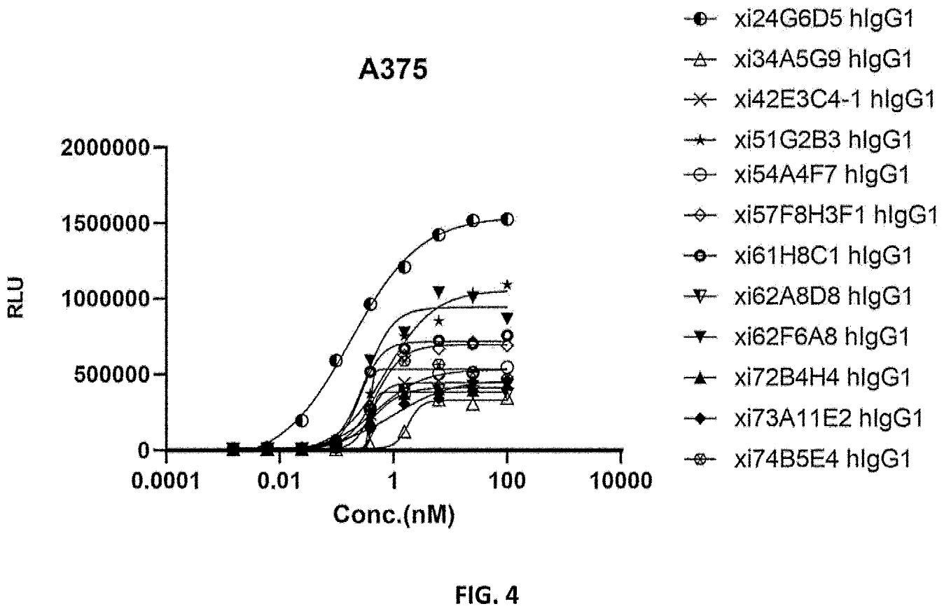

The results are presented in FIG. 4 and Table 5. As shown, all these chimeric antibodies exhibited very strong ADCC activities.

TABLE 5
Binding Affinity of Chimeric Antibodies

| Antibody | EC₅₀ (nM) | Top (MFI) |
|---|---|---|
| xi24G6D5 hIgG1 | 0.18 | 1543889 |
| xi34A5G9 hIgG1 | 1.83 | 329309 |
| xi42E3C4-1 hIgG1 | 0.38 | 445302 |
| xi51G2B3 hIgG1 | 0.75 | 1056643 |
| xi54A4F7 hIgG1 | 0.51 | 532808 |
| xi57F8H3F1 hIgG1 | 0.48 | 695892 |
| xi61H8C1 hIgG1 | 0.26 | 716855 |
| xi62A8D8 hIgG1 | 0.32 | 943767 |
| xi62F6A8 hIgG1 | ~0.4233 | 384827 |
| xi72B4H4 hIgG1 | 0.43 | 412773 |
| xi73A11E2 hIgG1 | 1.02 | 464841 |
| xi74B5E4 hIgG1 | ~0.3929 | 534397 |

### Example 6. Cytotoxic Activities of the Chimeric Antibodies

This example measured the cell killing activity of the chimeric antibodies.

A375 cells that endogenously expressed IL-13RA2 were seeded to a 96-well plate at 3000 cells per well. After overnight incubation, each diluted sample which has been conjugated with MMAE was added. After 5 days incubation, cell viability was measured by CellTiter-Glo reagent. The luciferase activity was detected by Envison.

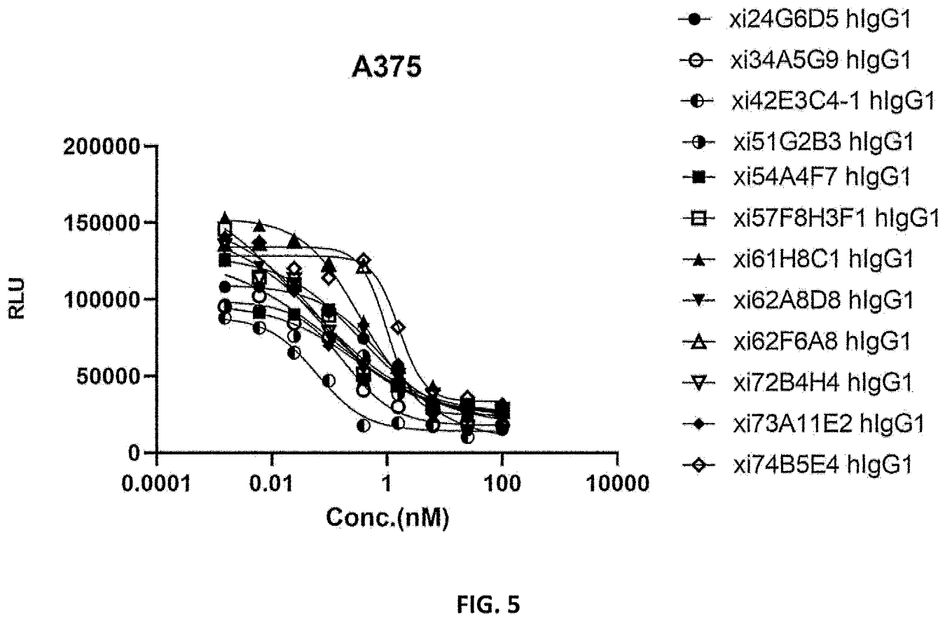

As shown in FIG. 5 and Table 6, these chimeric antibodies all have very strong cytotoxic activities.

TABLE 6
Cytotoxic Activity of Chimeric Antibodies

| Antibody | IC₅₀ (nM) | Top (MFI) |
|---|---|---|
| xi24G6D5 hIgG1 | 1.00 | 109775 |
| xi34A5G9 hIgG1 | 0.19 | 98834 |
| xi42E3C4-1 hIgG1 | 0.06 | 88587 |
| xi51G2B3 hIgG1 | 0.27 | 96866 |
| xi54A4F7 hIgG1 | 0.11 | 130274 |
| xi57F8H3F1 hIgG1 | 0.07 | 152191 |
| xi61H8C1 hIgG1 | 0.33 | 152919 |
| xi62A8D8 hIgG1 | 1.01 | 134240 |
| xi62F6A8 hIgG1 | 0.36 | 128503 |
| xi72B4H4 hIgG1 | 0.08 | 146238 |
| xi73A11E2 hIgG1 | 0.03 | 175106 |
| xi74B5E4 hIgG1 | 1.62 | 128406 |

### Example 6. Endocytosis of the Chimeric Antibodies

This example texted the chimeric antibodies for their ability to induce internalization upon binding to surface IL-13RA2.

The method uses a new hydrophilic and bright pH sensor dye (pHAb dye), which is not fluorescent at neutral pH but becomes highly fluorescent at acidic pH with internalization. It can be used to detect the process of endocytosis. CHO-K1/H_IL-13RA2, HEK-293/H IL-13RA2 cells which overexpress human IL-13RA2, and A375 endogenously expressed human IL-13RA2 as the target cells, the detection antibody labeled with pHAb dye was added to evaluate the endocytosis of IL-13RA2 chimeric monoclonal antibody in vitro.

Serial dilutions of IL-13RA2 chimeric monoclonal antibodies were incubated for 24 hours at 37° C. Luciferase activity was detected.

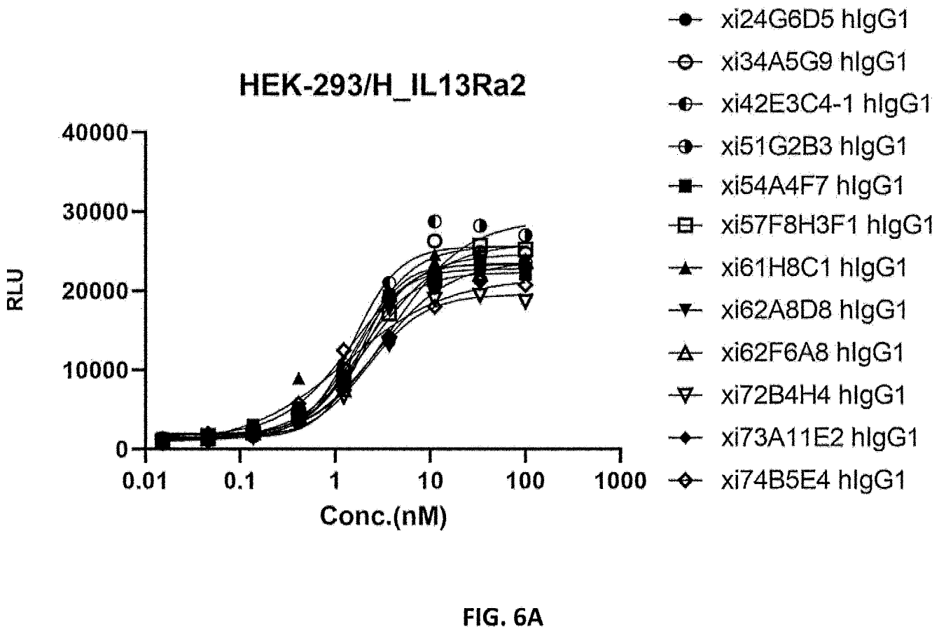

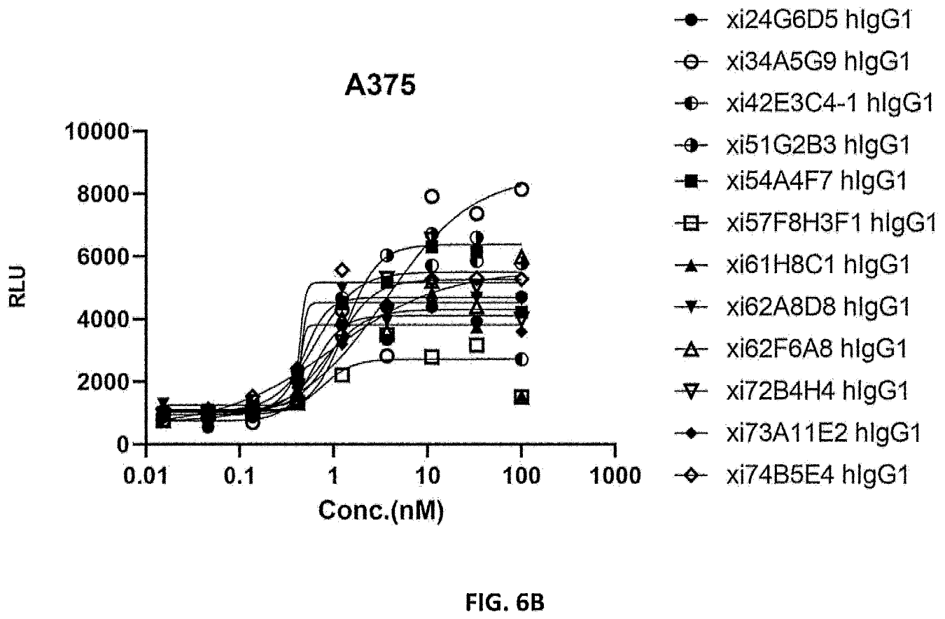

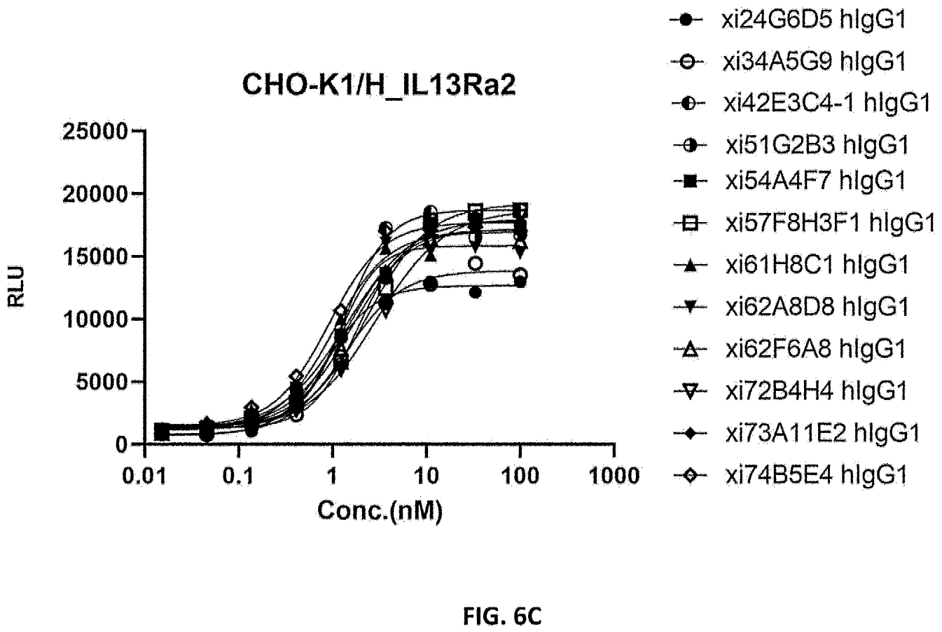

The results are shown in FIG. 6A-C and Table 7, which demonstrate that these chimeric antibodies have very strong endocytosis activities.

TABLE 7
Internalization of Chimeric Antibodies

| | 293 | 293 | CHO-K1 | CHO-K1 | A375 | A375 |
|---|---|---|---|---|---|---|
| Antibody | EC₅₀ (nM) | Top (MFI) | EC₅₀ (nM) | Top (MFI) | EC₅₀ (nM) | Top (MFI) |
| xi24G6D5 hIgG1 | 2.76 | 23713 | 0.86 | 12681 | 0.61 | 4114 |
| xi34A5G9 hIgG1 | 1.78 | 25655 | 1.38 | 13863 | 3.63 | 8610 |
| xi42E3C4-1 hIgG1 | 1.56 | 25623 | 1.35 | 18711 | 0.49 | 4695 |
| xi51G2B3 hIgG1 | 3.84 | 28949 | 1.84 | 17110 | 1.32 | 6384 |
| xi54A4F7 hIgG1 | 1.65 | 22791 | 1.42 | 17996 | 0.66 | 5505 |
| xi57F8H3F1 hIgG1 | 2.42 | 26010 | 2.37 | 19197 | 0.74 | 2725 |
| xi61H8C1 hIgG1 | 1.49 | 24686 | 1.02 | 16949 | ~0.4412 | 3824 |
| xi62A8D8 hIgG1 | 1.88 | 23262 | 1.58 | 17204 | 1.00 | 5553 |
| xi62F6A8 hIgG1 | 1.73 | 22304 | 1.17 | 15857 | ~0.4473 | 4531 |
| xi72B4H4 hIgG1 | 2.18 | 19546 | 2.92 | 18705 | 1.06 | 5262 |
| xi73A11E2 hIgG1 | 1.98 | 23481 | 1.52 | 17880 | 0.77 | 4311 |
| xi74B5E4 hIgG1 | 1.31 | 21621 | 0.91 | 17727 | ~0.4370 | 5173 |

### Example 7. Humanization of Mouse Antibodies

Four of the murine antibodies, 61H8C1, 57F8H3F1, 72B4H4 and 73A11E2, were selected for humanization.

The amino acid sequences of the VH and VL of each murine antibody were compared against the available database of human Ig gene sequences to find the overall best-matching human germline Ig gene sequences. The CDRs of the murine antibodies were then grafted into the matched human sequences. The cDNA was synthesized and used to produce the humanized antibodies. Certain back mutations from the murine antibodies were then introduced back to the humanized antibodies. Certain amino acids were mutated to reduce the chance of post-translational modification.

The amino acid sequences of the humanized antibodies are provided in Tables 8-11 below.

### Humanized Sequences

### A. 61H8C1

TABLE 8A
Humanization of 61H8C1-VH

| Name | Sequence | SEQ ID NO: |
|---|---|---|
| 61H8C1 VH | QVQLQQSGAELARPGASVMLSCKASGYTFTSYWTQWIKQRPGQGL | 13 |
| | EGIGAIYPGDDDTSYNQKFKGKATLTADKSSSTAYMQLNSLTSED | |
| | SAVYYCARGATLLRPFPYWGQGTLVTVSA | |
| VH_1 (CDR | QVQLVQSGAEVKKPGASVKVSCKASGYTFTSYWTQWVRQAPGQGL | 54 |
| grafting) | EWMGAIYPGDDDTSYNQKFKGRVTMTRDTSTSTVYMELSSLRSED | |
| | TAVYYCARGATLLRPFPYWGQGTLVTVSS | |
| VH_2 | QVQLVQSGAEVAKPGASVKVSCKASGYTFTSYWTQWIRQAPGQGL | 55 |
| | EGMGAIYPGDDDTSYNQKFKGRVTMTADTSTSTVYMELSSLRSED | |
| | TAVYYCARGATLLRPFPYWGQGTLVTVSS | |
| VH_3 | QVQLVQSGAEVAKPGASVKVSCKASGYTFTSYWTQWIRQAPGQGL | 56 |
| | EGIGAIYPGDDDTSYNQKFKGRATLTADKSTSTVYMQLSSLRSED | |
| | TAVYYCARGATLLRPFPYWGQGTLVTVSS | |
| VH_4 | QVQLVQSGAEVAKPGASVKLSCKASGYTFTSYWTQWIKQAPGQGL | 57 |
| | EGIGAIYPGDDDTSYNQKFKGKATLTADKSTSTAYMQLSSLRSED | |
| | TAVYYCARGATLLRPFPYWGQGTLVTVSS | |

TABLE 8B
CDR Sequences

| CDR | Sequence | SEQ ID NO: |
|---|---|---|
| CDR-H1 | SYWTQ | 25 |
| CDR-H2 | AIYPGDDDTSYNQKFKG | 26 |
| CDR-H3 | GATLLRPFPY | 27 |

TABLE 8C
Humanization of 61H8C1-VL

| | | SEQ |
|---|---|---|
| | | ID |
| Name | Sequence | NO: |
| 61H8C1 VL | DTVMTQSHKFMSTSVGDRVSTTCKASQDVSTAVAWYQQKPGQSPK | 14 |
| | LLTYSASYRYTGVPDRFTGSGSGTDFTFTISSVQAEDLAVYSCQQ | |
| | HYSIPPTFGGGTKLEIK | |
| VL_1 (CDR | DIQMTQSPSSLSASVGDRVTITCKASQDVSTAVAWYQQKPGKAPK | 58 |
| grafting) | LLIYSASYRYTGVPSRFSGSGSGTDFTLTISSLQPEDFATYYCQQ | |
| | HYSIPPTFGQGTKLEIK | |
| VL 2 | DIQMTQSPSSLSTSVGDRVTITCKASQDVSTAVAWYQQKPGKSPK | 59 |
| | LLIYSASYRYTGVPSRFSGSGSGTDFTLTISSLQPEDFATYSCQQ | |
| | HYSIPPTFGQGTKLEIK | |
| VL 3 | DIQMTQSPSSLSTSVGDRVTITCKASQDVSTAVAWYQQKPGKSPK | 60 |
| | LLIYSASYRYTGVPDRFSGSGSGTDFTFTISSLQPEDFATYSCQQ | |
| | HYSIPPTFGGGTKLEIK | |
| VL_4 | DTVMTQSPSSLSTSVGDRVTITCKASQDVSTAVAWYQQKPGKSPK | 61 |
| | LLIYSASYRYTGVPDRFTGSGSGTDFTFTISSVQPEDFATYSCQQ | |
| | HYSIPPTFGGGTKLEIK | |

TABLE 8D
CDR Sequences

| CDR | Sequence | SEQ ID NO: |
|---|---|---|
| CDR-L1 | KASQDVSTAVA | 28 |
| CDR-L2 | SASYRYT | 29 |
| CDR-L3 | QQHYSTPPT | 30 |

TABLE 8E
Humanized antibodies

| | VH | | | |
|---|---|---|---|---|
| | 61H8C1 | 61H8C1 | 61H8C1 | 61H8C1 |
| VL | VH_1 | VH_2 | VH_3 | VH_4 |
| 61H8C1 VL_1 | H1L1 | H2L1 | H3L1 | H4L1 |
| 61H8C1 VL_2 | H1L2 | H2L2 | H3L2 | H4L2 |
| 61H8C1 VL_3 | H1L3 | H2L3 | H3L3 | H4L3 |
| 61H8C1 VL_4 | H1L4 | H2L4 | H3L4 | H4L4 |

### B. 57F8H3F1

TABLE 9A
Humanization of 57F8H3F1-VH

| Name | Sequence | SEQ ID NO: |
|---|---|---|
| 57F8H3F1 VH | QVQLQQPGAELVRPGASVKLSCKASGYTFTNYWMNWIKQRPEQGL | 11 |
| | EWIGRIDPYETETHYNQKFRDRAILTVDKSSSTAFMQLSGLTSED | |
| | SAVYYCARGYGHYYGVDYWGQGTSVTVSS | |
| VH_1 (CDR | QVQLVQSGAEVKKPGASVKVSCKASGYTFTNYWMNWVRQAPGQGL | 62 |
| grafting) | EWMGRIDAYETETHYNQKFRDRVTMTRDTSTSTVYMELSSLRSED | |
| | TAVYYCARGYGHYYGVDYWGQGTLVTVSS | |
| VH_2 | QVQLVQSGAEVVKPGASVKVSCKASGYTFTNYWMNWVRQAPGQGL | 63 |
| | EWIGRIDAYETETHYNQKFRDRVTMTVDTSTSTVYMELSSLRSED | |
| | TAVYYCARGYGHYYGVDYWGQGTLVTVSS | |
| VH_3 | QVQLVQSGAEVVKPGASVKLSCKASGYTFTNYWMNWVKQAPGQGL | 64 |
| | EWIGRIDAYETETHYNQKFRDRATLTVDKSTSTVYMELSSLRSED | |
| | TAVYYCARGYGHYYGVDYWGQGTLVTVSS | |
| VH_4 | QVQLVQSGAEVVKPGASVKLSCKASGYTFTNYWMNWVKQRPGQGL | 65 |
| | EWIGRIDAYETETHYNQKFRDRAILTVDKSTSTAYMELRSLRSED | |
| | TAVYYCARGYGHYYGVDYWGQGTLVTVSS | |

TABLE 9B
CDR Sequences

| CDR | Sequence | SEQ ID NO: |
|---|---|---|
| CDR-H1 | NYWMN | 31 |
| CDR-H2 | RIDPYETETHYNQKFRD | 49 |
| | RIDAYETETHYNQKFRD | 32 |
| CDR-H3 | GYGHYYGVDY | 33 |

TABLE 9C
Humanization of 57F8H3F1 - VL

| Name | Sequence | SEQ ID NO: |
|---|---|---|
| 57F8H3F1 VL | DIVLTQSPASLAVSLGQRATISCRASENVGNFGISFVNWFQQKPG | 12 |
| | QPPKLLIYAASNLGSGVPARFSGSGSGTDFSLNIHPMEEDDAAMY | |
| | FCQQSKGVPWTFGGGTKLETK | |
| VL_1 (CDR | DIQMTQSPSSLSASVGDRVTITCRASENVGNFGISFVNWYQQKPG | 66 |
| grafting) | KAPKLLIYAASNLGSGVPSRFSGSGSGTDFTLTISSLQPEDFATY | |
| | YCQQSKGVPWTFGQGTKVEIK | |
| VL_2 | DIQMTQSPSSLSASVGDRVTITCRASENVGNFGISFVNWFQQKPG | 67 |
| | KAPKLLIYAASNLGSGDPSRFSGSGSGTDFTLTISSLQPEDFATY | |
| | FCQQSKGVPWTFGQGTKVEIK | |
| VL_3 | DIQLTQSPSSLSASVGDRVTITCRASENVGNFGISFVNWFQQKPG | 68 |
| | KAPKLLIYAASNLGSGDPARFSGSGSGTDFTLTISSLQPEDFATY | |
| | FCQQSKGVPWTFGGGTKVEIK | |
| VL_4 | DIVLTQSPSSLSASVGDRATITCRASENVGNFGTSFVNWFQQKPG | 69 |
| | KPPKLLIYAASNLGSGDPARFSGSGSGTDFTLTISSLQPEDFATY | |
| | FCQQSKGVPWTFGGGTKLEIK | |

TABLE 9D
CDR Sequences

| CDR | Sequence | SEQ ID NO: |
|---|---|---|
| CDR-L1 | RASENVGNFGISFVN | 34 |
| CDR-L2 | AASNLGS | 35 |
| CDR-L3 | QQSKGVPWT | 36 |

TABLE 9E
Humanized antibodies

| | VH | | | |
|---|---|---|---|---|
| | 57F8H3F1 | 57F8H3F1 | 57F8H3F1 | 57F8H3F1 |
| VL | VH_1 | VH_2 | VH_3 | VH_4 |
| 57F8H3F1 VL_1 | H1L1 | H2L1 | H3L1 | H4L1 |
| 57F8H3F1 VL_2 | H1L2 | H2L2 | H3L2 | H4L2 |
| 57F8H3F1 VL_3 | H1L3 | H2L3 | H3L3 | H4L3 |
| 57F8H3F1 VL_4 | H1L4 | H2L4 | H3L4 | H4L4 |

### C. 72B4H4

TABLE 10A
Humanization of 72B4H4-VH

| Name | Sequence | SEQ ID NO: |
|---|---|---|
| 72B4H4 VH | QVQLQQPGAERVRPGASVKLSCKASGYTFTNYWMNWVKQRPEQGL | 19 |
| | EWIGRIDPYAGETHYNQKFKDKAILTVDKSSSTAYMQLRSLTSED | |
| | SAVYYCARGYGDSYGMDHWGQGTSVTVSS | |
| VH_1 (CDR | QVQLVQSGAEVKKPGASVKVSCKASGYTFTNYWMNWVRQAPGQGL | 70 |
| grafting) | EWMGRIDAYAGETHYNQKFKDRVTMTRDTSTSTVYMELSSLRSED | |
| | TAVYYCARGYGDAYGMDHWGQGTLVTVSS | |
| VH_2 | QVQLVQSGAEVVKPGASVKVSCKASGYTFTNYWMNWVRQAPGQGL | 71 |
| | EWIGRIDAYAGETHYNQKFKDRVTMTVDTSTSTVYMELSSLRSED | |
| | TAVYYCARGYGDAYGMDHWGQGTLVTVSS | |
| VH_3 | QVQLVQSGAEVVKPGASVKLSCKASGYTFTNYWMNWVKQAPGQGL | 72 |
| | EWIGRIDAYAGETHYNQKFKDRATLTVDKSTSTVYMELSSLRSED | |
| | TAVYYCARGYGDAYGMDHWGQGTLVTVSS | |
| VH_4 | QVQLVQSGAEVVKPGASVKLSCKASGYTFTNYWMNWVKQRPGQGL | 73 |
| | EWIGRIDAYAGETHYNQKFKDRAILTVDKSTSTAYMELRSLRSED | |
| | TAVYYCARGYGDAYGMDHWGQGTLVTVSS | |

TABLE 10B
CDR Sequences

| CDR | Sequence | SEQ ID NO: |
|---|---|---|
| CDR-H1 | NYWMN | 37 |
| CDR-H2 | RIDPYAGETHYNQKFKD | 50 |
| | RIDAYAGETHYNQKFKD | 38 |
| CDR-H3 | GYGDSYGMDH | 51 |
| | GYGDAYGMDH | 39 |

TABLE 10C
Humanization of 72B4H4-VL

| Name | Sequence | SEQ |
|---|---|---|
| | | ID |
| | | NO: |
| 72B4H4 VL | DIVLTQSPASLAVSLGQRATISCRASESVDNYGISFMNWFQQKPG | 20 |
| | QPPKLLIYAASRQGSGDPARFSGSGSGTDFSLNIHPLEEDDTAMY | |
| | FCQQSKEVPWTFGGGTKLEIK | |
| VL_1 (CDR | DIQMTQSPSSLSASVGDRVTITCRASESVDNYGISFMNWYQQKPG | 74 |
| grafting) | KAPKLLIYAASRQGSGVPSRFSGSGSGTDFTLTISSLQPEDFATY | |
| | YCQQSKEVPWTFGQGTKVEIK | |
| VL_2 | DIQMTQSPSSLSASVGDRVTITCRASESVDNYGISFMNWFQQKPG | 75 |
| | KAPKLLIYAASRQGSGDPSRFSGSGSGTDFTLTISSLQPEDFATY | |
| | FCQQSKEVPWTFGQGTKVEIK | |
| VL_3 | DIQLTQSPSSLSASVGDRVTITCRASESVDNYGISFMNWFQQKPG | 76 |
| | KAPKLLIYAASRQGSGDPARFSGSGSGTDFTLTISSLQPEDFATY | |
| | FCQQSKEVPWTFGGGTKVEIK | |
| VL_4 | DTVLTQSPSSLSASVGDRATTTCRASESVDNYGISFMNWFQQKPG | 77 |
| | KPPKLLIYAASRQGSGDPARFSGSGSGTDFTLTISSLQPEDFATY | |
| | FCQQSKEVPWTFGGGTKLEIK | |

TABLE 10D
CDR Sequences

| CDR | Sequence | SEQ ID NO: |
|---|---|---|
| CDR-L1 | RASESVDNYGISFMN | 40 |
| CDR-L2 | AASRQGS | 41 |
| CDR-L3 | QQSKEVPWT | 42 |

TABLE 10E
Humanized antibodies

| | VH | | | |
|---|---|---|---|---|
| | 72B4H4 | 72B4H4 | 72B4H4 | 72B4H4 |
| VL | VH_1 | VH_2 | VH_3 | VH_4 |
| 72B4H4 VL_1 | H1L1 | H2L1 | H3L1 | H4L1 |
| 72B4H4 VL_2 | H1L2 | H2L2 | H3L2 | H4L2 |
| 72B4H4 VL_3 | H1L3 | H2L3 | H3L3 | H4L3 |
| 72B4H4 VL_4 | H1L4 | H2L4 | H3L4 | H4L4 |

### D. 73A11E2

TABLE 11A
Humanization of 73A11E2-VH

| Name | Sequence | SEQ ID NO: |
|---|---|---|
| 73A11E2 VH | EVKLVESGGGLVKPGGSLKLSCAASGFTFSSYGMSWVRQTPEKRL | 21 |
| | EWVATISGGGSYTYYPDSVKGRFTISRDNARNNLYLQMNSLRSED | |
| | TALYYCARRDLRSAMDYWGQGTSVTVSS | |
| VH_1 (CDR | QVQLVQSGAEVKKPGASVKVSCKASGFTFSSYGMSWVRQAPGQGL | 78 |
| grafting) | EWMGTISGGGSYTYYPESVKGRVTMTTDTSTSTAYMELRSLRSDD | |
| | TAVYYCARRDLRSAMDYWGQGTLVTVSS | |
| VH_2 | QVQLVQSGAEVVKPGASVKVSCKASGFTFSSYGMSWVRQAPGQGL | 79 |
| | EWVGTISGGGSYTYYPESVKGRFTITRDTSTSTAYMELRSLRSDD | |
| | TAVYYCARRDLRSAMDYWGQGTLVTVSS | |
| VH_3 | QVQLVQSGAEVVKPGASVKLSCKASGFTFSSYGMSWVRQAPGQGL | 80 |
| | EWVATISGGGSYTYYPESVKGRFTITRDNSTSTLYMEMRSLRSDD | |
| | TAVYYCARRDLRSAMDYWGQGTLVTVSS | |
| VH_4 | QVKLVESGAEVVKPGASVKLSCKASGFTFSSYGMSWVRQAPGQRL | 81 |
| | EWVATISGGGSYTYYPESVKGRFTITRDNSTSTLYLEMRSLRSDD | |
| | TAVYYCARRDLRSAMDYWGQGTLVTVSS | |

TABLE 11B
CDR Sequences

| CDR | Sequence | SEQ ID NO: |
|---|---|---|
| CDR-H1 | SYGMS | 43 |
| CDR-H2 | TISGGGSYTYYPDSVKG | 52 |
| | TISGGGSYTYYPESVKG | 44 |
| CDR-H3 | RDLRSAMDY | 45 |

TABLE 11C
Humanization of 73A11E2-VL

| Name | Sequence | SEQ ID NO: |
|---|---|---|
| 73A11E2 | DIVMTQSQKFMSTSVGDRVSVTCKASQNVDINVAWYQQKPGQSPK | 22 |
| VL | ALIYSASYRYSGVPDRFTGSGSGTDFTLTISNVQSEDLAEFFCQQ | |
| | YNSYPLTFGGGTKLEIK | |
| VL_1 (CDR | DVVMTQSPLSLPVTLGQPASISCKASQNVDINVAWFQQRPGQSPR | 82 |
| grafting) | RLIYSASYRYSGVPDRFSGSGSGTDFTLKISRVEAEDVGVYYCQQ | |
| | YNAYPLTFGQGTKLEIK | |
| VL_2 | DIVMTQSPLSLPVTLGQPASISCKASQNVDINVAWYQQRPGQSPR | 83 |
| | ALIYSASYRYSGVPDRFSGSGSGTDFTLKISRVEAEDVGVFYCQQ | |
| | YNAYPLTFGQGTKLEIK | |
| VL_3 | DIVMTQSPLSLPTTLGQPASISCKASQNVDINVAWYQQRPGQSPR | 84 |
| | ALIYSASYRYSGVPDRFSGSGSGTDFTLTISRVEAEDVGVFYCQQ | |
| | YNAYPLTFGGGTKLEIK | |
| VL_4 | DIVMTQSPLSLPTTLGQPVSVSCKASQNVDINVAWYQQRPGQSPR | 85 |
| | ALTYSASYRYSGVPDRFSGSGSGTDFTLTISRVEAEDVAVFFCQQ | |
| | YNAYPLTFGGGTKLEIK | |

TABLE 11D
CDR Sequences

| CDR | Sequence | SEQ ID NO: |
|---|---|---|
| CDR-L1 | KASQNVDINVA | 46 |
| CDR-L2 | SASYRYS | 47 |
| CDR-L3 | QQYNSYPLT | 53 |
| | QQYNAYPLT | 48 |

TABLE 11E
Humanized antibodies

| | VH | | | |
|---|---|---|---|---|
| | 73A11E2 | 73A11E2 | 73A11E2 | 73A11E2 |
| VL | VH_1 | VH_2 | VH_3 | VH_4 |
| 73A11E2 VL_1 | H1L1 | H2L1 | H3L1 | H4L1 |
| 73A11E2 VL_2 | H1L2 | H2L2 | H3L2 | H4L2 |
| 73A11E2 VL_3 | H1L3 | H2L3 | H3L3 | H4L3 |
| 73A11E2 VL_4 | H1L4 | H2L4 | H3L4 | H4L4 |

### Example 8. Binding Affinity of Humanized Antibodies

This example tested the binding affinity of the humanized antibodies.

A375 cells that endogenously expressed IL-13RA2, were harvested from flasks. 100 μl of 1×10⁶ cells/ml of cells were incubated with primary humanized antibodies in 4-fold serial dilutions starting from 200 nM to 0.003 nM for 30 minutes on ice. After being washed with 200 μl of FACS buffer twice, cells were incubated with secondary antibody for 30 minutes on ice. Cells were washed with 200 μl of FACS buffer twice and analyzed by FACS.

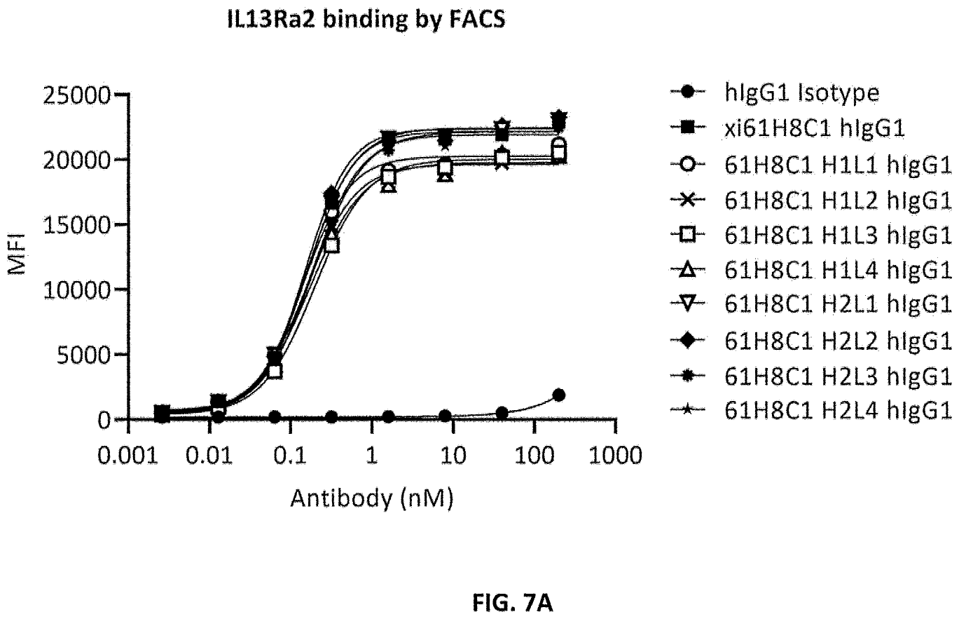

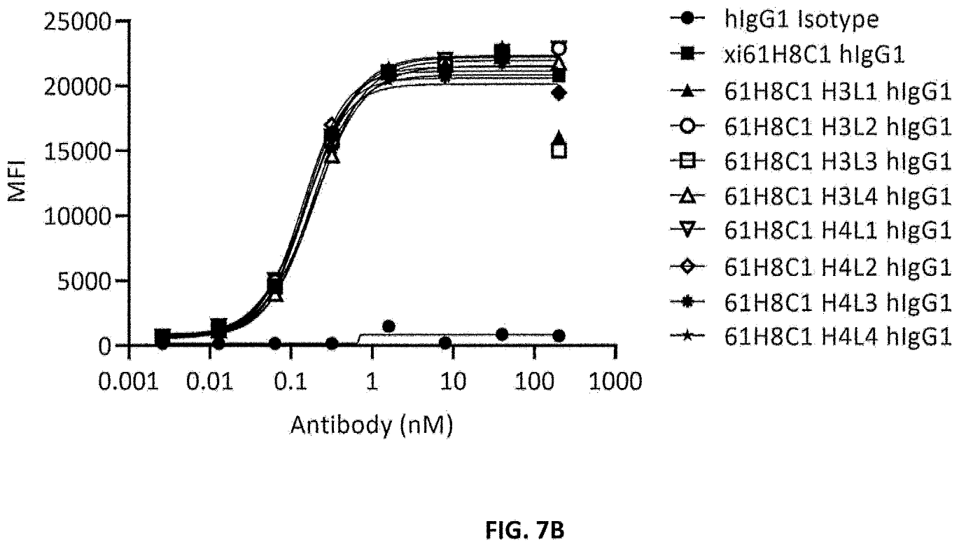

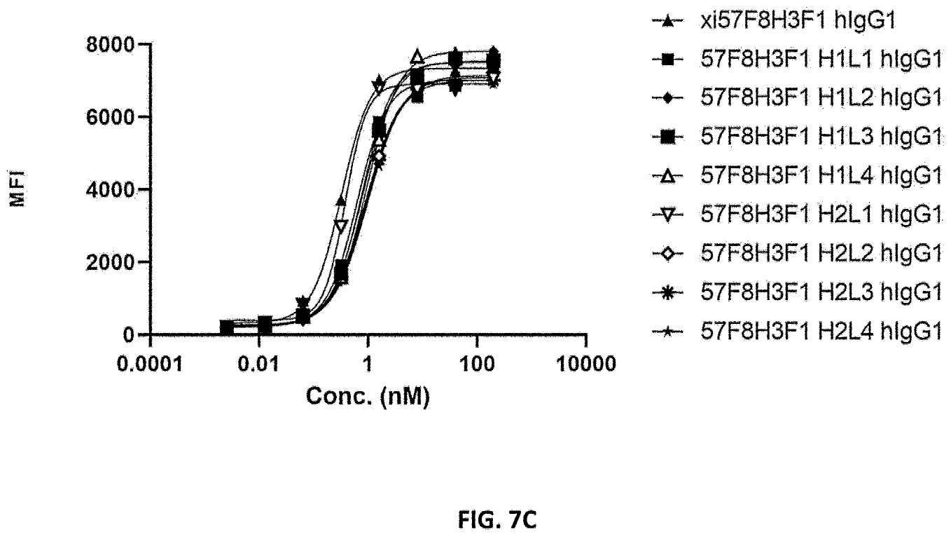

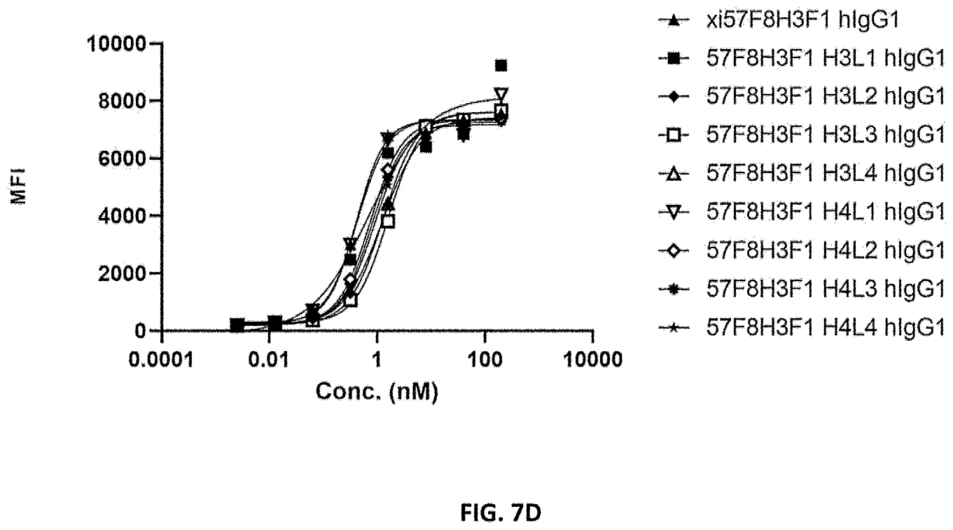

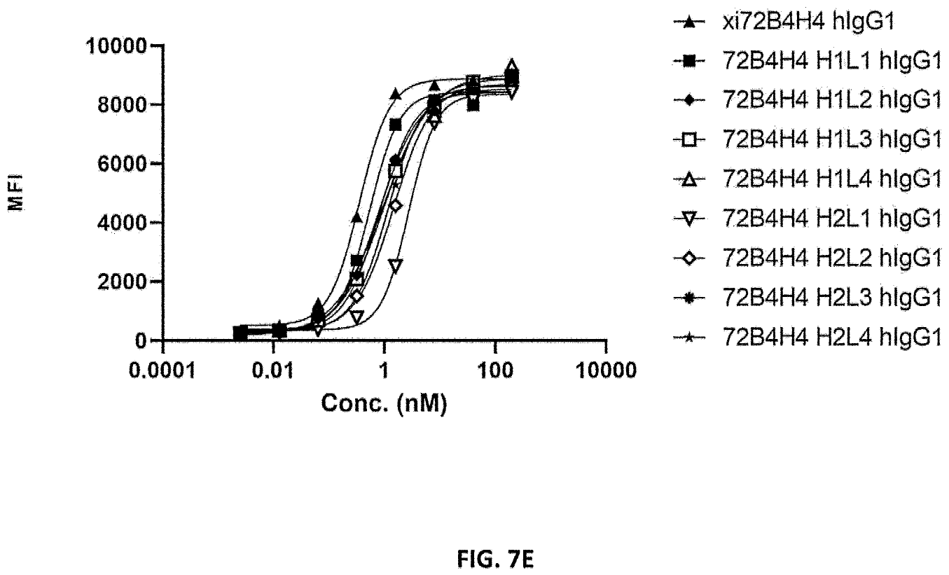

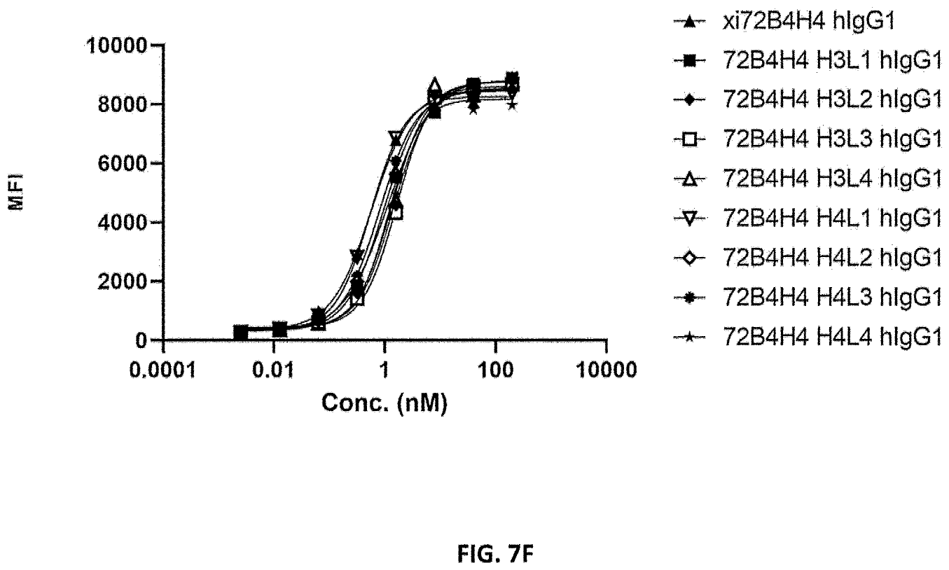

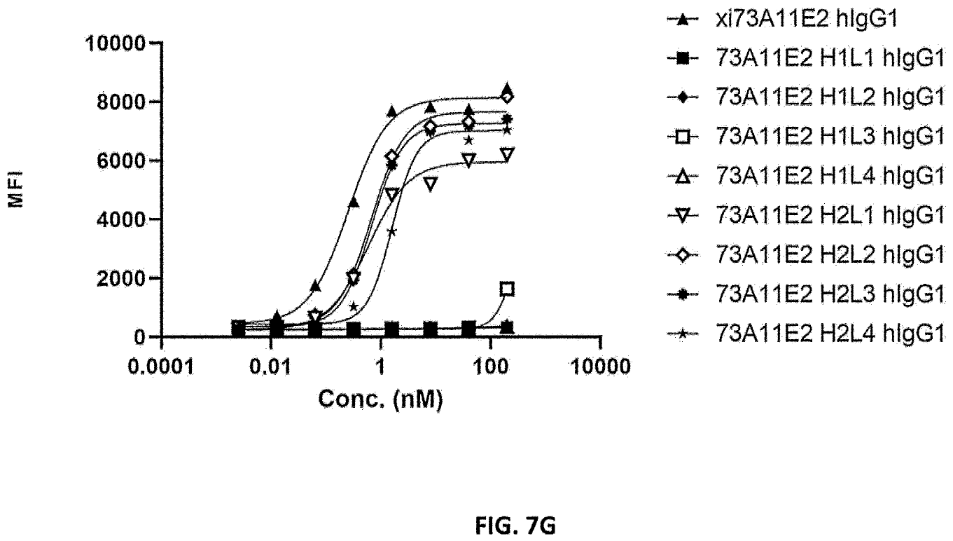

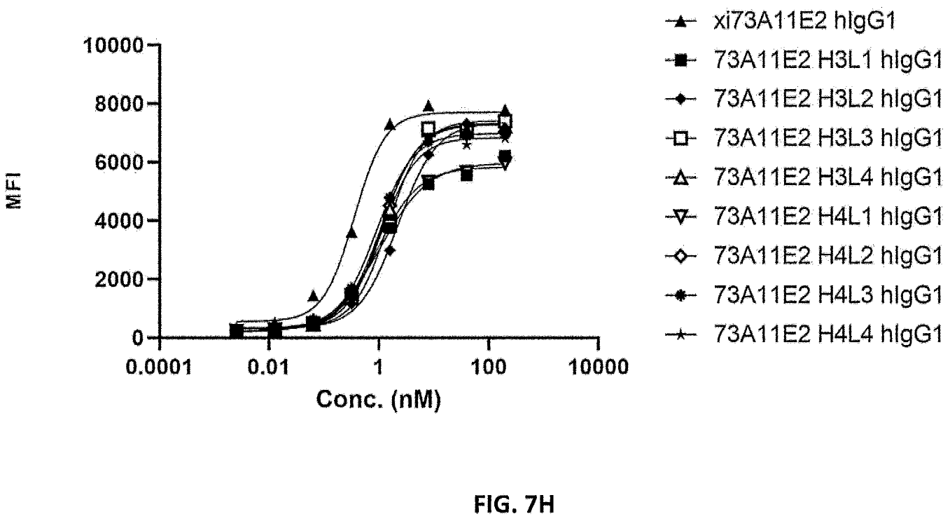

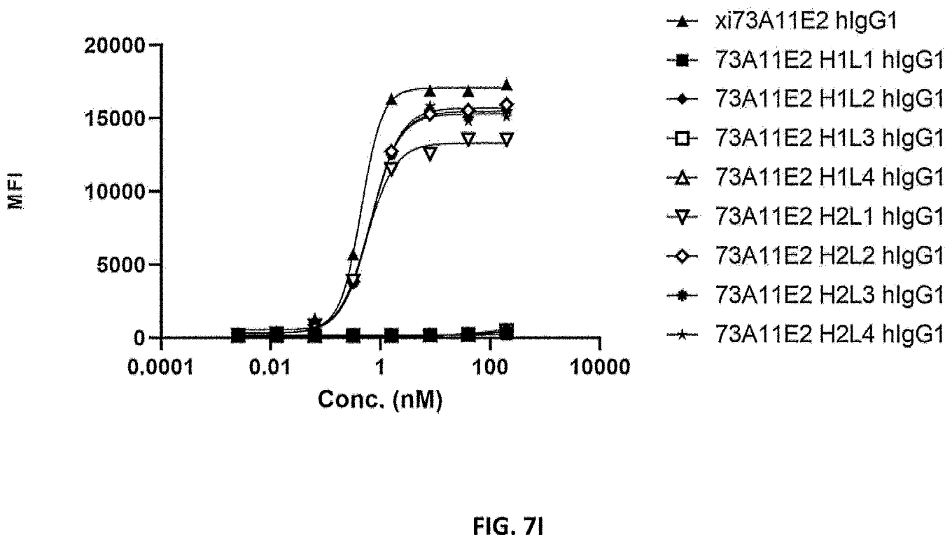

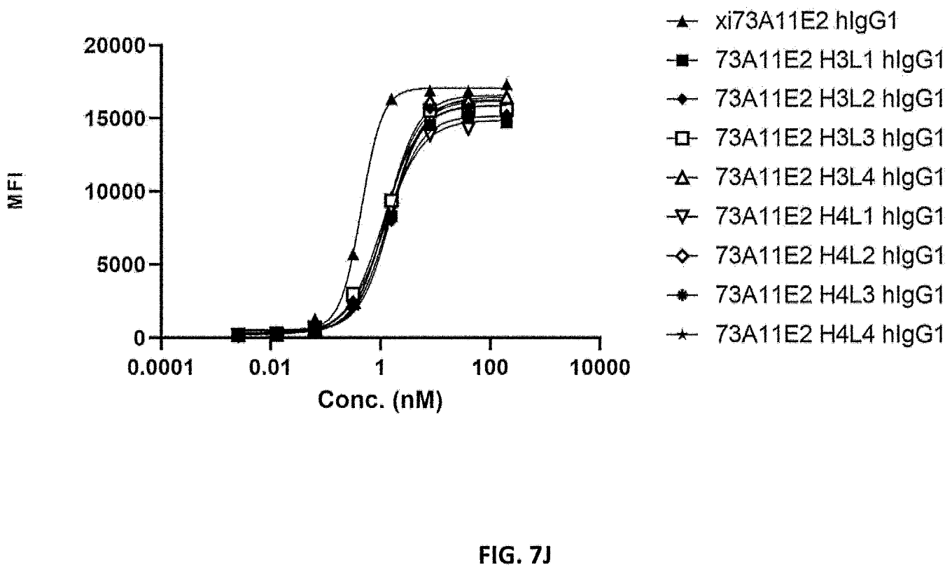

The results are presented in FIG. 7A- and Table 12. As shown, the humanized antibodies can bind to human IL-13RA2 at high EC50.

TABLE 12
A. Binding Affinity of Humanized Versions of 61H8C1

| Antibody | EC₅₀ (nM) | Top (MFI) |
|---|---|---|
| hIgG1 Isotype | N.A. | N.A. |
| xi61H8C1 hIgG1 | 0.16 | 22167 |
| 61H8C1 H1L1 hIgG1 | 0.14 | 20253 |
| 61H8C1 H1L2 hIgG1 | 0.15 | 19637 |
| 61H8C1 H1L3 hIgG1 | 0.20 | 20025 |
| 61H8C1 H1L4 hIgG1 | 0.17 | 19776 |
| 61H8C1 H2L1 hIgG1 | 0.16 | 22421 |
| 61H8C1 H2L2 hIgG1 | 0.15 | 22358 |
| 61H8C1 H2L3 hIgG1 | 0.19 | 22140 |
| 61H8C1 H2L4 hIgG1 | 0.18 | 21925 |

B. Binding Affinity of Humanized Versions of 61H8C1 (cont'd)

| Antibody | EC₅₀ (nM) | Top (MFI) |
|---|---|---|
| hIgG1 Isotype | N.A. | N.A. |
| xi61H8C1 hIgG1 | 0.16 | 21465 |
| 61H8C1 H3L1 hIgG1 | 0.15 | 20615 |
| 61H8C1 H3L2 hIgG1 | 0.17 | 22336 |
| 61H8C1 H3L3 hIgG1 | 0.15 | 20148 |
| 61H8C1 H3L4 hIgG1 | 0.20 | 21962 |
| 61H8C1 H4L1 hIgG1 | 0.17 | 22219 |
| 61H8C1 H4L2 hIgG1 | 0.14 | 21148 |
| 61H8C1 H4L3 hIgG1 | 0.18 | 20846 |
| 61H8C1 H4L4 hIgG1 | 0.19 | 21550 |

C. Binding Affinity of Humanized Versions of 57F8H3F1

| Antibody | EC₅₀ (nM) | Top (MFI) |
|---|---|---|
| xi57F8H3F1 hIgG1 | 0.33 | 7344 |
| 57F8H3F1 H1L1 hIgG1 | 0.63 | 7015 |
| 57F8H3F1 H1L2 hIgG1 | 0.78 | 7509 |
| 57F8H3F1 H1L3 hIgG1 | 0.81 | 7536 |
| 57F8H3F1 H1L4 hIgG1 | 0.94 | 7815 |
| 57F8H3F1 H2L1 hIgG1 | 0.38 | 6910 |
| 57F8H3F1 H2L2 hIgG1 | 0.89 | 7090 |
| 57F8H3F1 H2L3 hIgG1 | 0.92 | 7013 |
| 57F8H3F1 H2L4 hIgG1 | 1.01 | 7144 |

D. Binding Affinity of Humanized Versions of 57F8H3F1 (cont'd)

| Antibody | EC₅₀ (nM) | Top (MFI) |
|---|---|---|
| xi57F8H3F1 hIgG1 | 0.41 | 7340 |
| 57F8H3F1 H3L1 hIgG1 | 0.71 | 8157 |
| 57F8H3F1 H3L2 hIgG1 | 1.25 | 7424 |
| 57F8H3F1 H3L3 hIgG1 | 1.62 | 7614 |
| 57F8H3F1 H3L4 hIgG1 | 1.30 | 7609 |
| 57F8H3F1 H4L1 hIgG1 | 0.42 | 7353 |
| 57F8H3F1 H4L2 hIgG1 | 0.75 | 7335 |
| 57F8H3F1 H4L3 hIgG1 | 0.80 | 7184 |
| 57F8H3F1 H4L4 hIgG1 | 0.91 | 7267 |

E. Binding Affinity of Humanized Versions of 72B4H4

| Antibody | EC₅₀ (nM) | Top (MFI) |
|---|---|---|
| xi72B4H4 hIgG1 | 0.36 | 8877 |
| 72B4H4 H1L1 hIgG1 | 0.54 | 8438 |
| 72B4H4 H1L2 hIgG1 | 0.81 | 8536 |
| 72B4H4 H1L3 hIgG1 | 1.00 | 8902 |
| 72B4H4 H1L4 hIgG1 | 0.98 | 9045 |
| 72B4H4 H2L1 hIgG1 | 2.75 | 8357 |
| 72B4H4 H2L2 hIgG1 | 1.48 | 8699 |
| 72B4H4 H2L3 hIgG1 | 0.83 | 8420 |
| 72B4H4 H2L4 hIgG1 | 1.18 | 8634 |

F. Binding Affinity of Humanized Versions of 72B4H4 (cont'd)

| Antibody | EC₅₀ (nM) | Top (MFI) |
|---|---|---|
| xi72B4H4 hIgG1 | 0.5813 | 8499 |
| 72B4H4 H3L1 hIgG1 | 1.137 | 8829 |
| 72B4H4 H3L2 hIgG1 | 1.436 | 8551 |
| 72B4H4 H3L3 hIgG1 | 1.662 | 8777 |
| 72B4H4 H3L4 hIgG1 | 1.363 | 8769 |
| 72B4H4 H4L1 hIgG1 | 0.5522 | 8266 |
| 72B4H4 H4L2 hIgG1 | 0.9756 | 8613 |
| 72B4H4 H4L3 hIgG1 | 0.8109 | 8467 |
| 72B4H4 H4L4 hIgG1 | 1.183 | 8181 |

G. Binding Affinity of Humanized Versions of 73A11E2

| Antibody | EC₅₀ (nM) | Top (MFI) |
|---|---|---|
| xi73A11E2 hIgG1 | 0.26 | 8139 |
| 73A11E2 H1L1 hIgG1 | 82.75 | 389.7 |
| 73A11E2 H1L2 hIgG1 | ~23774557481 | ~412109 |
| 73A11E2 H1L3 hIgG1 | ~250.6 | ~4149 |
| 73A11E2 H1L4 hIgG1 | ~2.678e+017 | ~294650 |
| 73A11E2 H2L1 hIgG1 | 0.6 | 5960 |
| 73A11E2 H2L2 hIgG1 | 0.67 | 7664 |
| 73A11E2 H2L3 hIgG1 | 0.69 | 7269 |
| 73A11E2 H2L4 hIgG1 | 1.61 | 7019 |

H. Binding Affinity of Humanized Versions of 73A11E2 (cont'd)

| Antibody | EC₅₀ (nM) | Top (MFI) |
|---|---|---|
| xi73A11E2 hIgG1 | 0.3586 | 7701 |
| 73A11E2 H3L1 hIgG1 | 1.072 | 5980 |
| 73A11E2 H3L2 hIgG1 | 2.224 | 7368 |
| 73A11E2 H3L3 hIgG1 | 1.494 | 7423 |
| 73A11E2 H3L4 hIgG1 | 1.157 | 7285 |
| 73A11E2 H4L1 hIgG1 | 0.9434 | 5835 |
| 73A11E2 H4L2 hIgG1 | 1.122 | 7347 |
| 73A11E2 H4L3 hIgG1 | 0.9092 | 6985 |
| 73A11E2 H4L4 hIgG1 | 1.117 | 6839 |

I. Binding Affinity of Humanized Versions of 73A11E2 (cont'd)

| Antibody | EC₅₀ (nM) | Top (MFI) |
|---|---|---|
| xi73A11E2 hIgG1 | 0.45 | 17083 |
| 73A11E2 H1L1 hIgG1 | ~708625 | ~49273 |
| 73A11E2 H1L2 hIgG1 | 178.4 | 1029 |
| 73A11E2 H1L3 hIgG1 | ~521.7 | ~1490 |
| 73A11E2 H1L4 hIgG1 | ~388.5 | 996.9 |
| 73A11E2 H2L1 hIgG1 | 0.56 | 13314 |
| 73A11E2 H2L2 hIgG1 | 0.67 | 15705 |
| 73A11E2 H2L3 hIgG1 | 0.67 | 15470 |
| 73A11E2 H2L4 hIgG1 | 0.66 | 15306 |

J. Binding Affinity of Humanized Versions of 73A11E2 (cont'd)

| Antibody | EC₅₀ (nM) | Top (MFI) |
|---|---|---|
| xi73A11E2 hIgG1 | 0.45 | 17083 |
| 73A11E2 H3L1 hIgG1 | 1.37 | 15156 |
| 73A11E2 H3L2 hIgG1 | 1.53 | 15878 |
| 73A11E2 H3L3 hIgG1 | 1.17 | 15883 |
| 73A11E2 H3L4 hIgG1 | 1.29 | 16583 |
| 73A11E2 H4L1 hIgG1 | 1.20 | 14883 |
| 73A11E2 H4L2 hIgG1 | 1.31 | 16290 |
| 73A11E2 H4L3 hIgG1 | 1.45 | 16196 |
| 73A11E2 H4L4 hIgG1 | 1.51 | 16454 |

### Example 9. ADCC of the Humanized Antibodies

This example measured the antibody-dependent cellular cytotoxicity (ADCC) of the humanized antibodies.

The ADCC Reporter Bioassay uses an alternative readout at an earlier point in ADCC MOA pathway activation: the activation of gene transcription through the NFAT (nuclear factor of activated T-cells) pathway in the effector cell. In addition, the ADCC Reporter Bioassay uses engineered Jurkat cells stably expressing the FcγRIIIa receptor, V158 (high affinity) variant, and an NFAT response element driving expression of firefly luciferase as effector cells. Antibody biological activity in ADCC MOA is quantified through the luciferase produced as a result of NFAT pathway activation; luciferase activity in the effector cell is quantified with luminescence readout. Signal is high, and assay background is low.

Serial dilutions of IL-13RA2 humanized monoclonal antibody were incubated for 6 hours of induction at 37° C. with engineered Jurkat effector cells (ADCC Bioassay Effector Cells), with ADCC Bioassay Target Cells (A375 cell line). Luciferase activity was quantified using Bio-Glo™ Reagent.

The results show that these humanized antibodies have very strong ADCC activities.

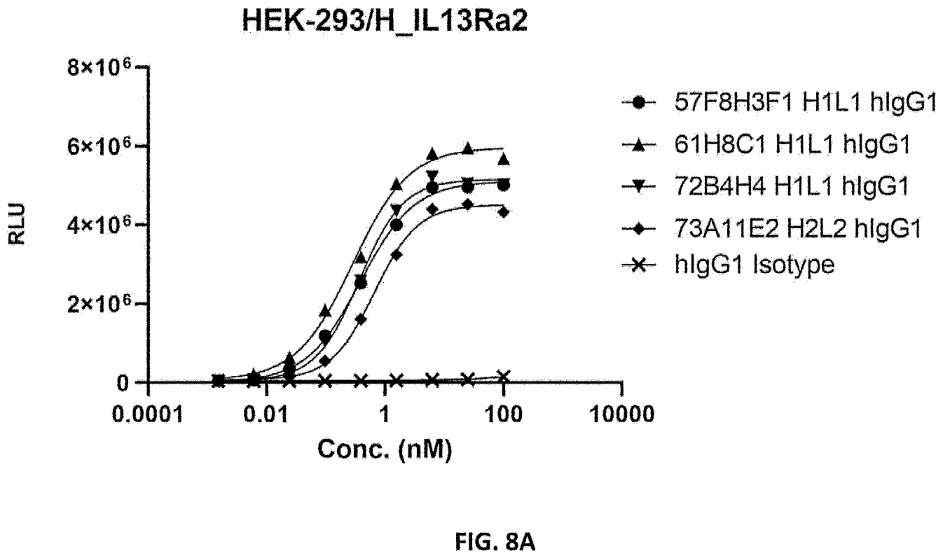

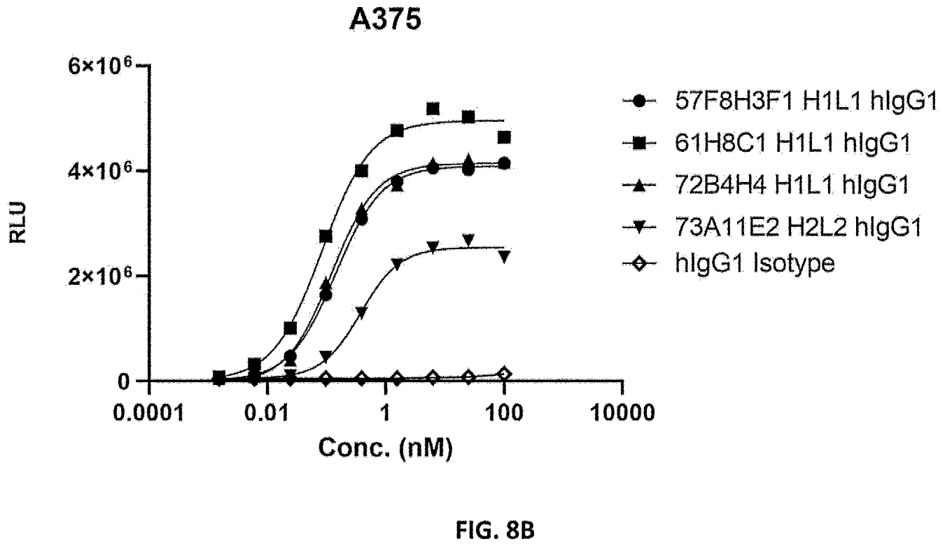

The results are presented in FIG. 8A (IL-13RA2 overexpression cell lines) and 8B (Endogenous Gliomas cell lines). As shown, all these humanized antibodies exhibited very strong ADCC activities.

### Example 10. Endocytosis of the Humanized Antibodies

This example texted the chimeric antibodies for their ability to induce internalization upon binding to surface IL-13RA2.

The method uses a new hydrophilic and bright pH sensor dye (pHAb dye), which is not fluorescent at neutral pH but becomes highly fluorescent at acidic pH with internalization. It can be used to detect the process of endocytosis. CHO-K1/H_IL-13RA2, HEK-293/H IL-13RA2 cells which overexpress human IL-13RA2, and A375 endogenously expressed human IL-13RA2 as the target cells, the detection antibody labeled with pHAb dye was added to evaluate the endocytosis of IL-13RA2 chimeric monoclonal antibody in vitro.

Serial dilutions of selected humanized antibodies were incubated for 24 hours at 37° C. Luciferase activity was detected.

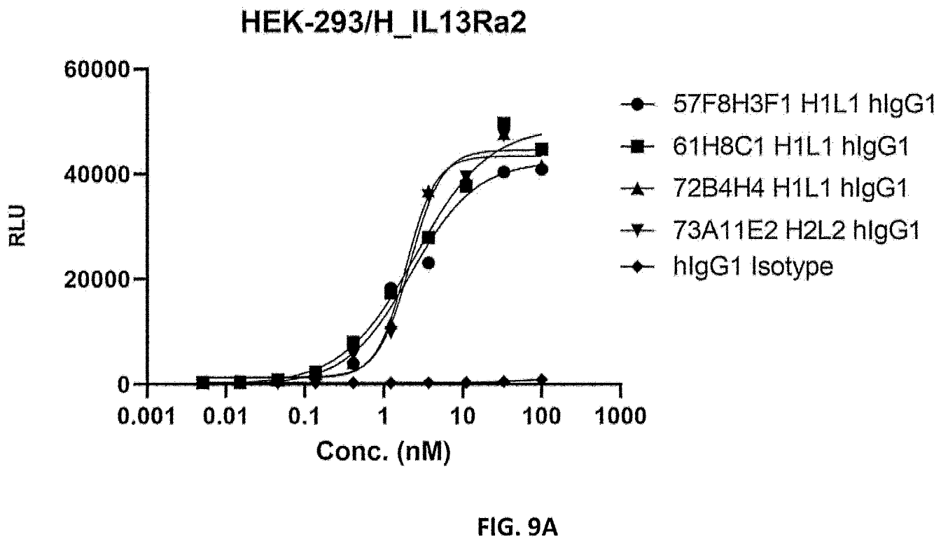

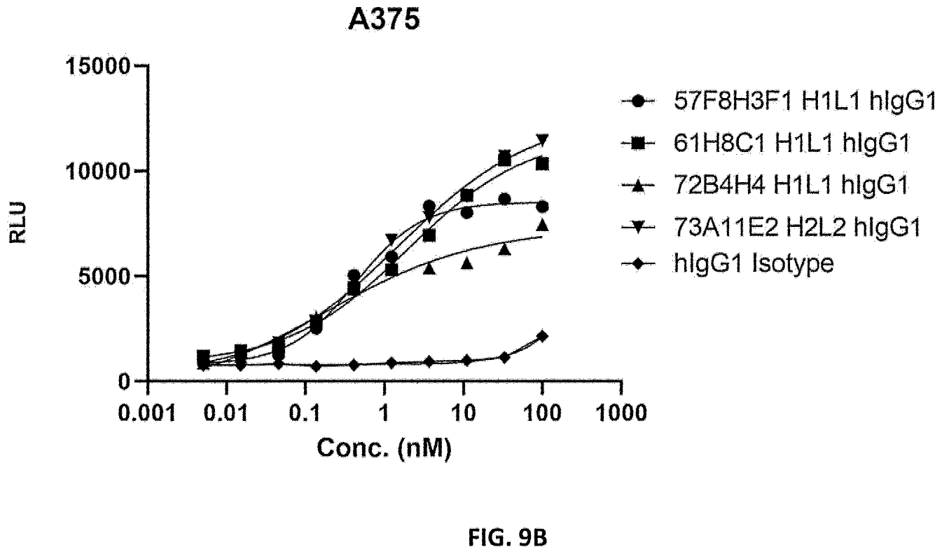

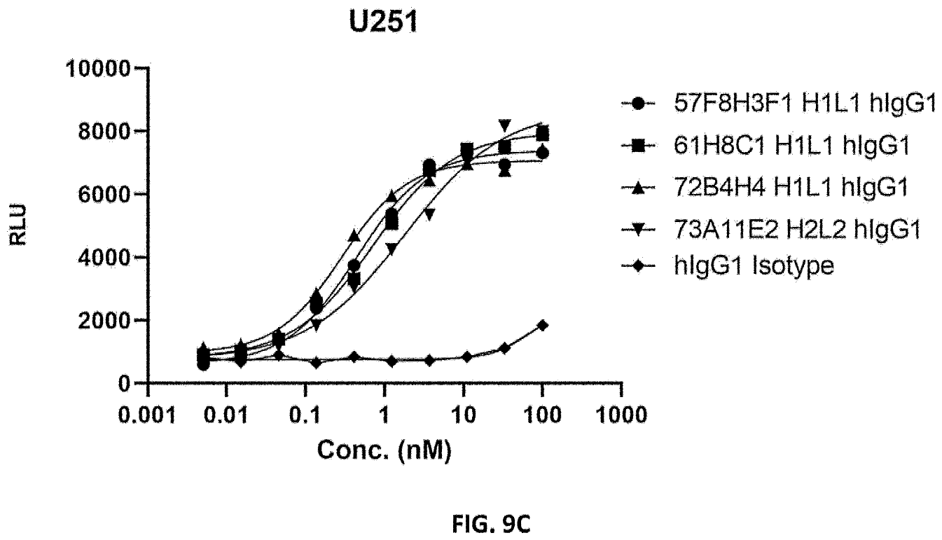

The results are shown in FIG. 9A (IL-13RA2 overexpression cell lines), and 9B-C (endogenous cell lines), which demonstrate that these humanized antibodies have very strong endocytosis activities.

### Example 11. In Vitro Cytotoxicity of Antibody-Drug Conjugates

This example tested the cytotoxicity of humanized antibodies conjugated to toxic drugs (ADC) on different Gliomas cells.

A375 and U251 cells that endogenously expressed IL-13RA2, HEK-293 cells that stably expressed IL-13RA2 were seeded to a 96-well plate at 3000 cells per well. After overnight incubation, each diluted sample which has been conjugated with MMAE was added. After 5 days incubation, cell viability was measured by CellTiter-Glo reagent. The luciferase activity was detected by Envison.

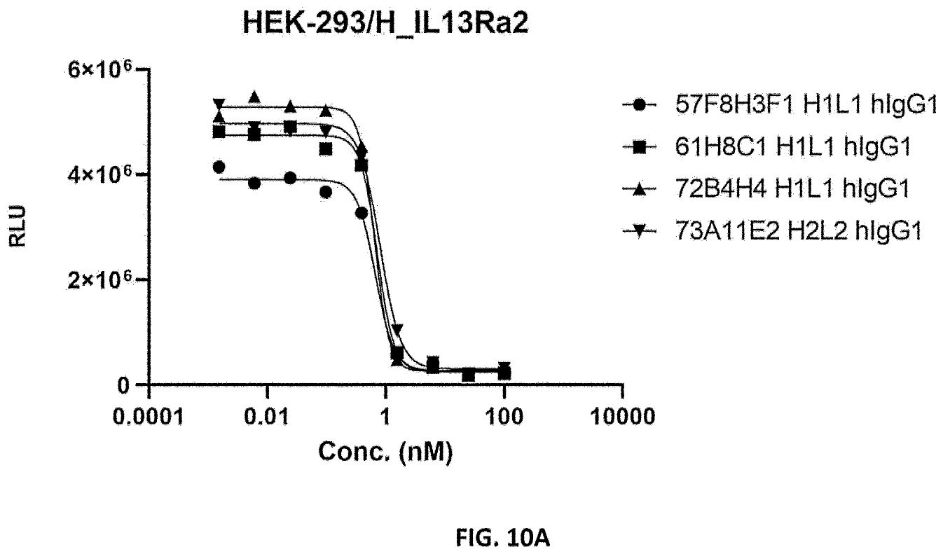

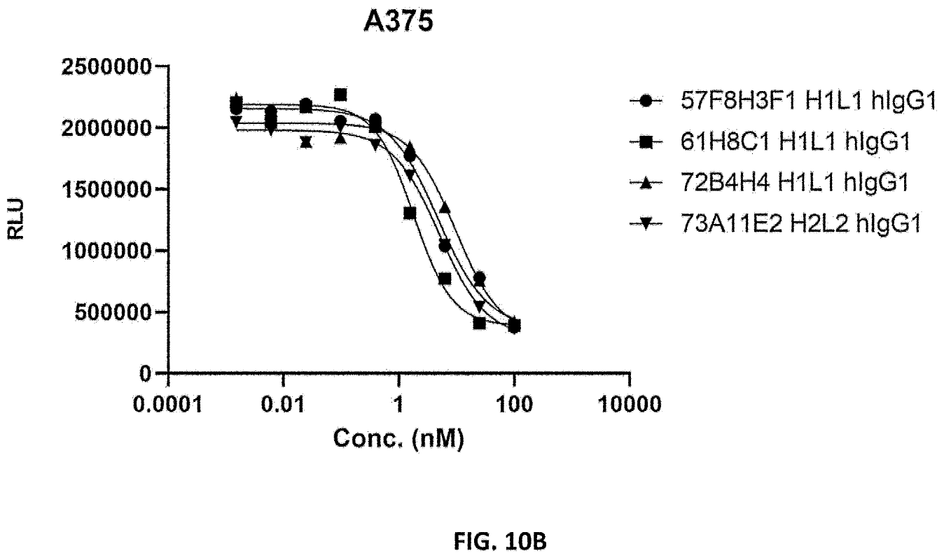

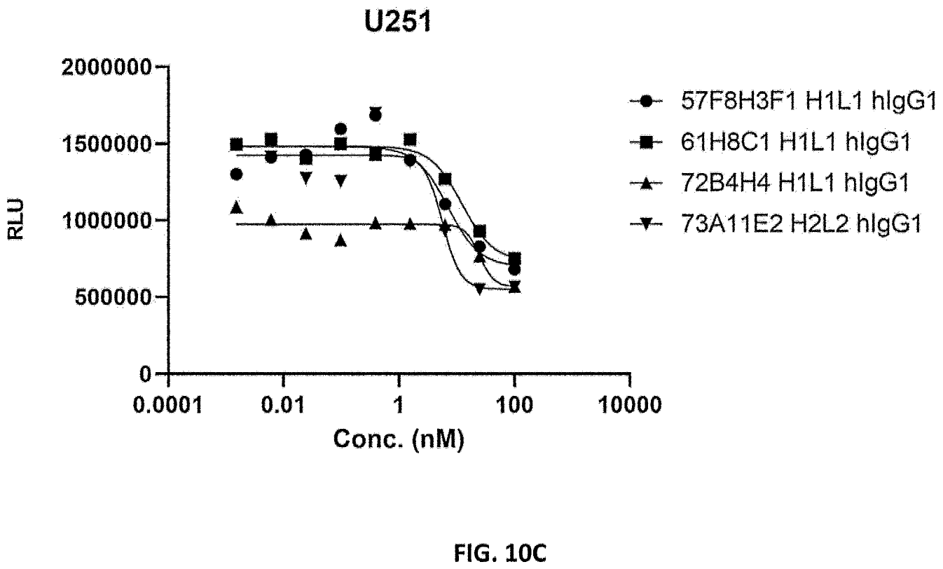

As shown in FIG. 10A-C, these ADC have very strong cytotoxic activities.

### Example 12. In Vivo Cytotoxicity of Antibody-Drug Conjugates

This example tested the in vivo cytotoxicity of humanized antibodies conjugated to MMAE (ADC).

In this study, 6-8 weeks female NCG mice (Jiangsu Jicui Yaokang Biotechnology Co., Ltd) were used. Each mouse was inoculated subcutaneously at the right axillary (lateral) with A375 tumor cell (5×106) in 0.1 ml of PBS with matrigel (V:V=1:1) for tumor development. The animals were grouped when tumor volume reach around 70 mm³, which is the 5th day after cell inoculation, then treatment started for the efficacy study.

Testing articles at dose of 3 mg/kg was administrated via intravenous (i.v.) at day 0, day 7, and day 14. The experiment was terminated at day 23. Tumor sizes were measured three times weekly in two dimensions using a caliper and the volume was expressed in mm³ using the formula: V=0.5 a×b² where a and b were the long and short diameters of the tumor, respectively. The tumor sizes were then used for the calculations of T/C (%) values. T/C (%) was calculated using the formula: T/C %=(Ti−T0)/(Vi−V0)×100, Ti was the average tumor volume of a treatment group on a given day, T0 was the average tumor volume of the treatment group on the first day of treatment, Vi was the average tumor volume of the vehicle control group on the same day with Ti, and V0 was the average tumor volume of the vehicle group on the first day of treatment. TGI was calculated for each group using the formula: TGI (%)=[100−T/C]. Data points represent group (n=5) mean, error bars represent standard error of the mean (SEM). p value was calculated based on tumor size by t-test, compared with PBS control.

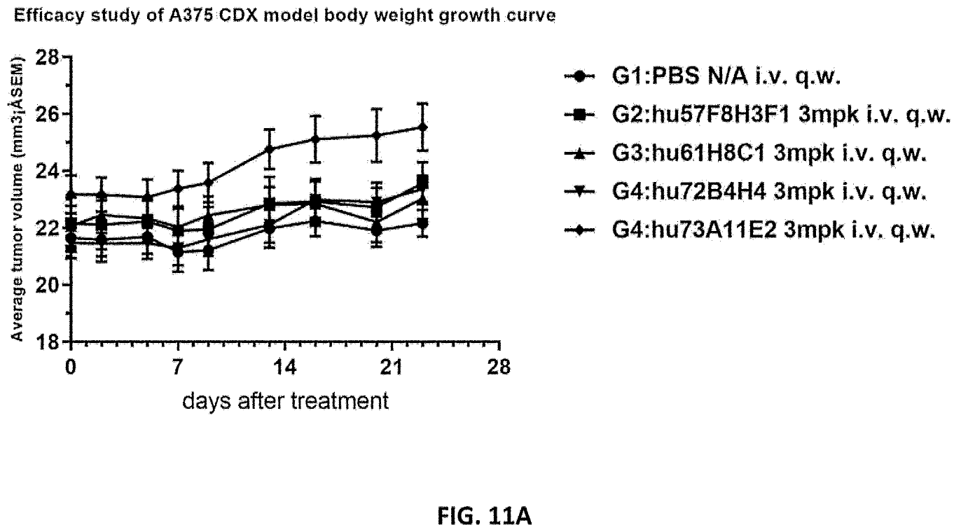

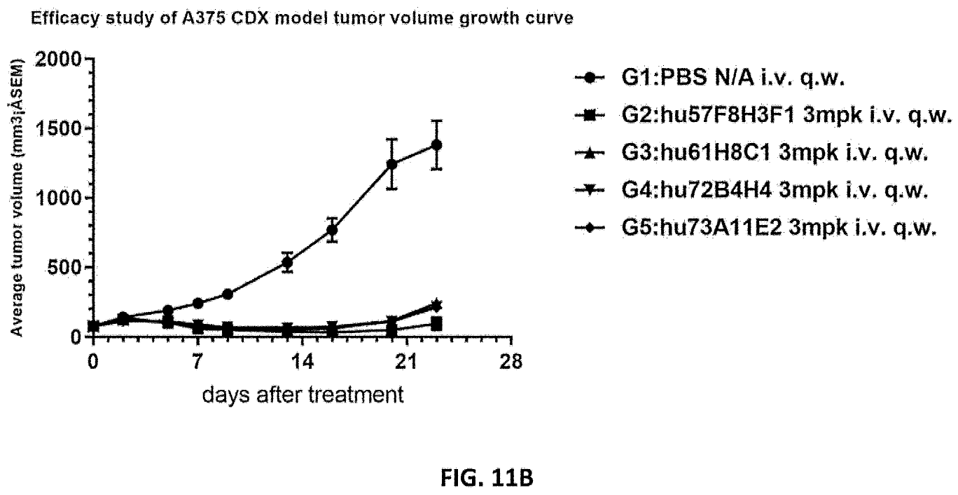

FIG. 11A shows body weight changes of the tumor-bearing mice in different groups. Data points represent group mean body weights. Error bars represent standard error of the mean (SEM). FIG. 11B and Table 13 show tumor growth curves of A375 tumor-bearing mice post administration of testing ADCs. As shown in these figures, the ADC exhibited strong in vivo cytotoxic activities.

TABLE 13
Tumor growth measurement post treatments

| | | Days after treatment | | | | | | | | |
|---|---|---|---|---|---|---|---|---|---|---|
| | | 0 | 2 | 5 | 7 | 9 | 13 | 16 | 20 | 23 |
| T/C | G2 | — | 71.58 | 23.20 | −10.55 | −10.69 | −8.24 | −6.18 | −2.29 | 1.37 |
| (%) | G3 | — | 98.78 | 21.32 | 7.86 | −3.80 | −1.39 | −0.72 | 3.21 | 12.67 |
| | G4 | — | 60.43 | 33.37 | 5.46 | −3.84 | −3.40 | −0.58 | 2.86 | 10.72 |
| | G5 | — | 84.85 | 22.37 | −0.35 | −4.15 | −4.71 | −2.48 | 3.39 | 10.43 |
| TGI | G2 | — | 28.42 | 76.80 | 110.55 | 110.69 | 108.24 | 106.18 | 102.29 | 98.63 |
| (%) | G3 | — | 1.22 | 78.68 | 92.14 | 103.80 | 101.39 | 100.72 | 96.79 | 87.33 |
| | G4 | — | 39.57 | 66.63 | 94.54 | 103.84 | 103.40 | 100.58 | 97.14 | 89.28 |
| | G5 | — | 15.15 | 77.63 | 100.35 | 104.15 | 104.71 | 102.48 | 96.61 | 89.57 |
| P | G2 | 0.9515 | 0.2847 | 0.0038 | 0.0001 | 0.0000 | 0.0001 | 0.0000 | 0.0002 | 0.0001 |
| value | G3 | 0.9733 | 0.9455 | 0.0009 | 0.0003 | 0.0000 | 0.0002 | 0.0000 | 0.0002 | 0.0002 |
| | G4 | 0.9704 | 0.1019 | 0.0033 | 0.0002 | 0.0000 | 0.0001 | 0.0000 | 0.0002 | 0.0002 |
| | G5 | 0.9061 | 0.5923 | 0.0014 | 0.0002 | 0.0000 | 0.0001 | 0.0000 | 0.0002 | 0.0002 |

The present disclosure is not to be limited in scope by the specific embodiments described which are intended as single illustrations of individual aspects of the disclosure, and any compositions or methods which are functionally equivalent are within the scope of this disclosure. It will be apparent to those skilled in the art that various modifications and variations can be made in the methods and compositions of the present disclosure without departing from the spirit or scope of the disclosure. Thus, it is intended that the present disclosure cover the modifications and variations of this disclosure provided they come within the scope of the appended claims and their equivalents.

All publications and patent applications mentioned in this specification are herein incorporated by reference to the same extent as if each individual publication or patent application was specifically and individually indicated to be incorporated by reference.

1. An antibody or fragment thereof having binding specificity to a human interleukin 13 receptor subunit alpha 2 (IL-13RA2) protein, wherein the antibody or fragment thereof comprises a heavy chain variable region comprising heavy chain complementarity determining regions CDRH1, CDRH2, and CDRH3 and a light chain variable region light chain comprising complementarity determining regions CDRL1, CDRL2, and CDRL3, and wherein:
(a) the CDRH1 comprises the amino acid sequence of SEQ ID NO: 25,
the CDRH2 comprises the amino acid sequence of SEQ ID NO: 26,
the CDRH3 comprises the amino acid sequence of SEQ ID NO: 27,
the CDRL1 comprises the amino acid sequence of SEQ ID NO: 28,
the CDRL2 comprises the amino acid sequence of SEQ ID NO: 29, and
the CDRL3 comprises the amino acid sequence of SEQ ID NO: 30,
(b) the CDRH1 comprises the amino acid sequence of SEQ ID NO: 31,
the CDRH2 comprises the amino acid sequence of SEQ ID NO: 32 or 49,
the CDRH3 comprises the amino acid sequence of SEQ ID NO: 33,
the CDRL1 comprises the amino acid sequence of SEQ ID NO: 34,
the CDRL2 comprises the amino acid sequence of SEQ ID NO: 35, and
the CDRL3 comprises the amino acid sequence of SEQ ID NO: 36,
(c) the CDRH1 comprises the amino acid sequence of SEQ ID NO: 37,
the CDRH2 comprises the amino acid sequence of SEQ ID NO: 38 or 50,
the CDRH3 comprises the amino acid sequence of SEQ ID NO: 39 or 51,
the CDRLI comprises the amino acid sequence of SEQ ID NO: 40,
the CDRL2 comprises the amino acid sequence of SEQ ID NO: 41, and
the CDRL3 comprises the amino acid sequence of SEQ ID NO: 42, or
(d) the CDRH1 comprises the amino acid sequence of SEQ ID NO: 43,
the CDRH2 comprises the amino acid sequence of SEQ ID NO: 44 or 52,
the CDRH3 comprises the amino acid sequence of SEQ ID NO: 45,
the CDRLI comprises the amino acid sequence of SEQ ID NO: 46,
the CDRL2 comprises the amino acid sequence of SEQ ID NO: 47, and
the CDRL3 comprises the amino acid sequence of SEQ ID NO: 48 or 53.

2. The antibody or fragment thereof of claim 1, wherein the CDRH1 comprises the amino acid sequence of SEQ ID NO: 25, the CDRH2 comprises the amino acid sequence of SEQ ID NO: 26, the CDRH3 comprises the amino acid sequence of SEQ ID NO: 27, the CDRLI comprises the amino acid sequence of SEQ ID NO: 28, the CDRL2 comprises the amino acid sequence of SEQ ID NO: 29, and the CDRL3 comprises the amino acid sequence of SEQ ID NO: 30.

3. The antibody or fragment thereof of claim 2, wherein the heavy chain variable region comprises an amino acid sequence selected from the group consisting of SEQ ID NO: 13 and 54-57, and the light chain variable region comprises an amino acid sequence selected from the group consisting of SEQ ID NO: 14 and 58-61.

4. The antibody or fragment thereof of claim 2, wherein the heavy chain variable region comprises the amino acid sequence of SEQ ID NO:54, and the light chain variable region comprises the amino acid sequence of SEQ ID NO:58.

5. The antibody or fragment thereof of claim 1, wherein the CDRHI comprises the amino acid sequence of SEQ ID NO: 31, the CDRH2 comprises the amino acid sequence of SEQ ID NO: 32 or 49, the CDRH3 comprises the amino acid sequence of SEQ ID NO: 33, the CDRL1 comprises the amino acid sequence of SEQ ID NO: 34, the CDRL2 comprises the amino acid sequence of SEQ ID NO: 35, and the CDRL3 comprises the amino acid sequence of SEQ ID NO: 36.

6. The antibody or fragment thereof of claim 5, wherein the CDRH2 comprises the amino acid sequence of SEQ ID NO: 32.

7. The antibody or fragment thereof of claim 5, wherein the heavy chain variable region comprises an amino acid sequence selected from the group consisting of SEQ ID NO: 11 and 62-65, and the light chain variable region comprises an amino acid sequence selected from the group consisting of SEQ ID NO: 12 and 66-69.

8. The antibody or fragment thereof of claim 5, wherein the heavy chain variable region comprises the amino acid sequence of SEQ ID NO:62, and the light chain variable region comprises the amino acid sequence of SEQ ID NO:66.

9. The antibody or fragment thereof of claim 1, wherein the CDRH1 comprises the amino acid sequence of SEQ ID NO: 37, the CDRH2 comprises the amino acid sequence of SEQ ID NO: 38 or 50, the CDRH3 comprises the amino acid sequence of SEQ ID NO: 39 or 51, the CDRL1 comprises the amino acid sequence of SEQ ID NO: 40, the CDRL2 comprises the amino acid sequence of SEQ ID NO: 41, and the CDRL3 comprises the amino acid sequence of SEQ ID NO: 42.

10. The antibody or fragment thereof of claim 9, wherein the CDRH2 comprises the amino acid sequence of SEQ ID NO: 38, and the CDRH3 comprises the amino acid sequence of SEQ ID NO: 39.

11. The antibody or fragment thereof of claim 9, wherein the heavy chain variable region comprises an amino acid sequence selected from the group consisting of SEQ ID NO: 19 and 70-73, and the light chain variable region comprises an amino acid sequence selected from the group consisting of SEQ ID NO: 20 and 74-77.

12. The antibody or fragment thereof of claim 9, wherein the heavy chain variable region comprises the amino acid sequence of SEQ ID NO:70, and the light chain variable region comprises the amino acid sequence of SEQ ID NO:74.

13. The antibody or fragment thereof of claim 1, wherein the CDRHI comprises the amino acid sequence of SEQ ID NO: 43, the CDRH2 comprises the amino acid sequence of SEQ ID NO: 44 or 52, the CDRH3 comprises the amino acid sequence of SEQ ID NO: 45, the CDRL1 comprises the amino acid sequence of SEQ ID NO: 46, the CDRL2 comprises the amino acid sequence of SEQ ID NO: 47, and the CDRL3 comprises the amino acid sequence of SEQ ID NO: 48 or 53.

14. The antibody or fragment thereof of claim 13, wherein the CDRH2 comprises the amino acid sequence of SEQ ID NO: 44, and the CDRL3 comprises the amino acid sequence of SEQ ID NO: 48.

15. The antibody or fragment thereof of claim 13, wherein the heavy chain variable region comprises an amino acid sequence selected from the group consisting of SEQ ID NO: 21 and 78-81, and the light chain variable region comprises an amino acid sequence selected from the group consisting of SEQ ID NO: 22 and 82-85.

16. The antibody or fragment thereof of claim 13, wherein the heavy chain variable region comprises the amino acid sequence of SEQ ID NO:79, and the light chain variable region comprises the amino acid sequence of SEQ ID NO:83.

17. (canceled)

18. (canceled)

19. The antibody or fragment of claim 1, which is capable of mediating antibody-dependent cellular cytotoxicity (ADCC).

20. (canceled)

21. An antibody-drug conjugate, comprising an antibody or fragment thereof of claim 1 conjugated to an antineoplastic agent.

22. (canceled)

23. One or more polynucleotide encoding the antibody or fragment thereof of claim 1.

24. (canceled)

25. A method of treating cancer or inflammation in a patient in need thereof, comprising administering to the patient the antibody or fragment thereof of claim 1.

26-28. (canceled)


SEQUENCE LISTING (85 sequences)

SEQ ID NO: 1 - protein, 115 aa, synthetic construct
QVQLQQSGPELVKPGTSVKMSCKASGYTFTSNYIHWVKQRPGQGLEWIGWIYPGDGSIKYNEKFKGKTTLTADKSSSTVYMLLSSLTSEDSAIYFCAVLRRLDYWGQGTTLTVSS

SEQ ID NO: 2 - protein, 107 aa, synthetic construct
DIVLTQSPATLSVTPGDSVSLSCRASQSISNNLHWYQQKSHESPRLLIKYASQSISGIPSRFSGSGSGTDFTLSINSVETEDFGMYFCQQSNSWPLTFGAGTKLELK

SEQ ID NO: 3 - protein, 119 aa, synthetic construct
EVHLQQSGPELVKPGASVKISCKTSGYTFTEYTMHWVKQSHGKSLEWIGRINPNNGIAEYKQTFKDKATLAVDKSSSTAYMEFRSLTSEDSAVYYCARGEGYGYWFAYWGQGTLVTVSA

SEQ ID NO: 4 - protein, 107 aa, synthetic construct
DIQMTQSPASLSASVGETVTITCRTSDNIYSYLAWYQQRQGKSPQLLVSNARTLAEGVPSRFSGSGSGTQFSLKINSLQPEDFGIYYCQHHSGTPFTFGSGTNLEIE

SEQ ID NO: 5 - protein, 118 aa, synthetic construct
EVKLVESGGGLVRPGGSLKISCAASGFTFSTYGMSWVRQTPEKRLEWVATISGGGSYTYYPDSVKGRFTISRDNAKNNLYLQMSSLRSEDTALYYCARRDLRSAMDYWGQGTSVTVSS

SEQ ID NO: 6 - protein, 107 aa, synthetic construct
DIVMTQSQKFMSTSVGDRVSVTCKASQNVDINVAWYQQKPGQSPKALIYSASFRYSGVPDRFTGSGSGTDFTLTISNVQSEDLAEYFCQQYNSYPLTFGGGTKLEIK

SEQ ID NO: 7 - protein, 119 aa, synthetic construct
QVQLQQSGPELVKPGASVKISCKASGYAFSDSWMNWVKQRPGQGLEWIGRIYPGDGDTNYNGKFKGKATLTADKSSNTAYMQLSSLTSVDSAVYFCARSWNGSPWFPYWGQGTLVTVSA

SEQ ID NO: 8 - protein, 112 aa, synthetic construct
DVLMTQTPLSLPVSLGDQASISCRSSQTIAHRNGNTYLEWYLQKPGQSPKLLLYKVSNRFSGVPDRFSGSGSGTDFTLKISRVEAEDLGVYYCFQGSHVTWTFGGGTKLEIK

SEQ ID NO: 9 - protein, 118 aa, synthetic construct
EVKLVESGGGLVKPGGSLKLSCAASGFTFSSYGMSWVRQTPEKRLEWVATISGGGSYTYYPDSVKGRFTISRDNAKNNLYLQMSSLRSEDTALYYCARRDLRSAMDHWGQGTSVTVSS

SEQ ID NO: 10 - protein, 107 aa, synthetic construct
DIVMTQSQKFMSTSVGDRVSVTCRASQNVDINVAWYQQKPGQSPKSLIYSASYRYSGVPDRFTGSGSGTDFTLTISNVQSEDLAEYFCQQYNSYPLTFGGGTKLEIR

SEQ ID NO: 11 - protein, 119 aa, synthetic construct
QVQLQQPGAELVRPGASVKLSCKASGYTFTNYWMNWIKQRPEQGLEWIGRIDPYETETHYNQKFRDRAILTVDKSSSTAFMQLSGLTSEDSAVYYCARGYGHYYGVDYWGQGTSVTVSS

SEQ ID NO: 12 - protein, 111 aa, synthetic construct
DIVLTQSPASLAVSLGQRATISCRASENVGNFGISFVNWFQQKPGQPPKLLIYAASNLGSGVPARFSGSGSGTDFSLNIHPMEEDDAAMYFCQQSKGVPWTFGGGTKLEIK

SEQ ID NO: 13 - protein, 119 aa, synthetic construct
QVQLQQSGAELARPGASVMLSCKASGYTFTSYWTQWIKQRPGQGLEGIGAIYPGDDDTSYNQKFKGKATLTADKSSSTAYMQLNSLTSEDSAVYYCARGATLLRPFPYWGQGTLVTVSA

SEQ ID NO: 14 - protein, 107 aa, synthetic construct
DIVMTQSHKFMSTSVGDRVSITCKASQDVSTAVAWYQQKPGQSPKLLIYSASYRYTGVPDRFTGSGSGTDFTFTISSVQAEDLAVYSCQQHYSIPPTFGGGTKLEIK

SEQ ID NO: 15 - protein, 118 aa, synthetic construct
EVKLVESGGGLVKPGGSLKLSCAASGFTFSSYGMSWVRQTPEKRLEWVATISGGGSYTYYPDSVKGRFTISRDNAKNNLYLQMSSLRSEDTALYYCARRDLRSAMDYWGQGTSVTVSS

SEQ ID NO: 16 - protein, 107 aa, synthetic construct
DIVMTQSQKFMSTSVGDRVSVTCKASQNVDINVAWYQQKPGQSPKALIYSASYRYSGVPDRFTGSGSGTDFTLTISNVQSEDLAEFFCQQYNNYPLTFGGGTKLEIK

SEQ ID NO: 17 - protein, 119 aa, synthetic construct
QVQIQQSGAELARPGASVMLSCKASGYTFTDYWMQWIKQRPGQGLEGIGAIYPGDGDTAYNQKFRGKATLTADKSSSTAYMQLNSLASEDSAVYFCARGATLLRPFPFWGQGTLVTVSA

SEQ ID NO: 18 - protein, 107 aa, synthetic construct
DIVMTQSHKFMSTSVGDRVSITCKASQDVSTAVAWYQQKPGQSPKLLIYSASYRYAGVPDRFTGSGSGTDFTFTISSVQAEDLAVYSCQQHYSIPPTFGGGTKLEIK

SEQ ID NO: 19 - protein, 119 aa, synthetic construct
QVQLQQPGAERVRPGASVKLSCKASGYTFTNYWMNWVKQRPEQGLEWIGRIDPYAGETHYNQKFKDKAILTVDKSSSTAYMQLRSLTSEDSAVYYCARGYGDSYGMDHWGQGTSVTVSS

SEQ ID NO: 20 - protein, 111 aa, synthetic construct
DIVLTQSPASLAVSLGQRATISCRASESVDNYGISFMNWFQQKPGQPPKLLIYAASRQGSGDPARFSGSGSGTDFSLNIHPLEEDDTAMYFCQQSKEVPWTFGGGTKLEIK

SEQ ID NO: 21 - protein, 118 aa, synthetic construct
EVKLVESGGGLVKPGGSLKLSCAASGFTFSSYGMSWVRQTPEKRLEWVATISGGGSYTYYPDSVKGRFTISRDNARNNLYLQMNSLRSEDTALYYCARRDLRSAMDYWGQGTSVTVSS

SEQ ID NO: 22 - protein, 107 aa, synthetic construct
DIVMTQSQKFMSTSVGDRVSVTCKASQNVDINVAWYQQKPGQSPKALIYSASYRYSGVPDRFTGSGSGTDFTLTISNVQSEDLAEFFCQQYNSYPLTFGGGTKLEIK

SEQ ID NO: 23 - protein, 118 aa, synthetic construct
EVKLVESGGGLVKPGGSLRLSCAASGFTFSTYGMSWVRQTPEKRLEWVATISGGGSYTYYPDSVKGRFTISRDNAKNNLYLQMSSLRSEDTALYYCARRDLRSAMDYWGQGTSVTVSS

SEQ ID NO: 24 - protein, 107 aa, synthetic construct
DIVMTQSQKFMSTSVGDRVSVTCKASQNVDINVAWYQQKPGQSPKALIYSASYRYSGVPDRFTGSGSGTDFTLTISNVQSEDLAEYFCQQYNTYPLTFGGGTKLEIK

SEQ ID NO: 25 - protein, 5 aa, synthetic construct
SYWTQ

SEQ ID NO: 26 - protein, 17 aa, synthetic construct
AIYPGDDDTSYNQKFKG

SEQ ID NO: 27 - protein, 10 aa, synthetic construct
GATLLRPFPY

SEQ ID NO: 28 - protein, 11 aa, synthetic construct
KASQDVSTAVA

SEQ ID NO: 29 - protein, 7 aa, synthetic construct
SASYRYT

SEQ ID NO: 30 - protein, 9 aa, synthetic construct
QQHYSIPPT

SEQ ID NO: 31 - protein, 5 aa, synthetic construct
NYWMN

SEQ ID NO: 32 - protein, 17 aa, synthetic construct
RIDAYETETHYNQKFRD

SEQ ID NO: 33 - protein, 10 aa, synthetic construct
GYGHYYGVDY

SEQ ID NO: 34 - protein, 15 aa, synthetic construct
RASENVGNFGISFVN

SEQ ID NO: 35 - protein, 7 aa, synthetic construct
AASNLGS

SEQ ID NO: 36 - protein, 9 aa, synthetic construct
QQSKGVPWT

SEQ ID NO: 37 - protein, 5 aa, synthetic construct
NYWMN

SEQ ID NO: 38 - protein, 17 aa, synthetic construct
RIDAYAGETHYNQKFKD

SEQ ID NO: 39 - protein, 10 aa, synthetic construct
GYGDAYGMDH

SEQ ID NO: 40 - protein, 15 aa, synthetic construct
RASESVDNYGISFMN

SEQ ID NO: 41 - protein, 7 aa, synthetic construct
AASRQGS

SEQ ID NO: 42 - protein, 9 aa, synthetic construct
QQSKEVPWT

SEQ ID NO: 43 - protein, 5 aa, synthetic construct
SYGMS

SEQ ID NO: 44 - protein, 17 aa, synthetic construct
TISGGGSYTYYPESVKG

SEQ ID NO: 45 - protein, 9 aa, synthetic construct
RDLRSAMDY

SEQ ID NO: 46 - protein, 11 aa, synthetic construct
KASQNVDINVA

SEQ ID NO: 47 - protein, 7 aa, synthetic construct
SASYRYS

SEQ ID NO: 48 - protein, 9 aa, synthetic construct
QQYNAYPLT

SEQ ID NO: 49 - protein, 17 aa, synthetic construct
RIDPYETETHYNQKFRD

SEQ ID NO: 50 - protein, 17 aa, synthetic construct
RIDPYAGETHYNQKFKD

SEQ ID NO: 51 - protein, 10 aa, synthetic construct
GYGDSYGMDH

SEQ ID NO: 52 - protein, 17 aa, synthetic construct
TISGGGSYTYYPDSVKG

SEQ ID NO: 53 - protein, 9 aa, synthetic construct
QQYNSYPLT

SEQ ID NO: 54 - protein, 119 aa, synthetic construct
QVQLVQSGAEVKKPGASVKVSCKASGYTFTSYWTQWVRQAPGQGLEWMGAIYPGDDDTSYNQKFKGRVTMTRDTSTSTVYMELSSLRSEDTAVYYCARGATLLRPFPYWGQGTLVTVSS

SEQ ID NO: 55 - protein, 119 aa, synthetic construct
QVQLVQSGAEVAKPGASVKVSCKASGYTFTSYWTQWIRQAPGQGLEGMGAIYPGDDDTSYNQKFKGRVTMTADTSTSTVYMELSSLRSEDTAVYYCARGATLLRPFPYWGQGTLVTVSS

SEQ ID NO: 56 - protein, 119 aa, synthetic construct
QVQLVQSGAEVAKPGASVKVSCKASGYTFTSYWTQWIRQAPGQGLEGIGAIYPGDDDTSYNQKFKGRATLTADKSTSTVYMQLSSLRSEDTAVYYCARGATLLRPFPYWGQGTLVTVSS

SEQ ID NO: 57 - protein, 119 aa, synthetic construct
QVQLVQSGAEVAKPGASVKLSCKASGYTFTSYWTQWIKQAPGQGLEGIGAIYPGDDDTSYNQKFKGKATLTADKSTSTAYMQLSSLRSEDTAVYYCARGATLLRPFPYWGQGTLVTVSS

SEQ ID NO: 58 - protein, 107 aa, synthetic construct
DIQMTQSPSSLSASVGDRVTITCKASQDVSTAVAWYQQKPGKAPKLLIYSASYRYTGVPSRFSGSGSGTDFTLTISSLQPEDFATYYCQQHYSIPPTFGQGTKLEIK

SEQ ID NO: 59 - protein, 107 aa, synthetic construct
DIQMTQSPSSLSTSVGDRVTITCKASQDVSTAVAWYQQKPGKSPKLLIYSASYRYTGVPSRFSGSGSGTDFTLTISSLQPEDFATYSCQQHYSIPPTFGQGTKLEIK

SEQ ID NO: 60 - protein, 107 aa, synthetic construct
DIQMTQSPSSLSTSVGDRVTITCKASQDVSTAVAWYQQKPGKSPKLLIYSASYRYTGVPDRFSGSGSGTDFTFTISSLQPEDFATYSCQQHYSIPPTFGGGTKLEIK

SEQ ID NO: 61 - protein, 107 aa, synthetic construct
DIVMTQSPSSLSTSVGDRVTITCKASQDVSTAVAWYQQKPGKSPKLLIYSASYRYTGVPDRFTGSGSGTDFTFTISSVQPEDFATYSCQQHYSIPPTFGGGTKLEIK

SEQ ID NO: 62 - protein, 119 aa, synthetic construct
QVQLVQSGAEVKKPGASVKVSCKASGYTFTNYWMNWVRQAPGQGLEWMGRIDAYETETHYNQKFRDRVTMTRDTSTSTVYMELSSLRSEDTAVYYCARGYGHYYGVDYWGQGTLVTVSS

SEQ ID NO: 63 - protein, 119 aa, synthetic construct
QVQLVQSGAEVVKPGASVKVSCKASGYTFTNYWMNWVRQAPGQGLEWIGRIDAYETETHYNQKFRDRVTMTVDTSTSTVYMELSSLRSEDTAVYYCARGYGHYYGVDYWGQGTLVTVSS

SEQ ID NO: 64 - protein, 119 aa, synthetic construct
QVQLVQSGAEVVKPGASVKLSCKASGYTFTNYWMNWVKQAPGQGLEWIGRIDAYETETHYNQKFRDRATLTVDKSTSTVYMELSSLRSEDTAVYYCARGYGHYYGVDYWGQGTLVTVSS

SEQ ID NO: 65 - protein, 119 aa, synthetic construct
QVQLVQSGAEVVKPGASVKLSCKASGYTFTNYWMNWVKQRPGQGLEWIGRIDAYETETHYNQKFRDRAILTVDKSTSTAYMELRSLRSEDTAVYYCARGYGHYYGVDYWGQGTLVTVSS

SEQ ID NO: 66 - protein, 111 aa, synthetic construct
DIQMTQSPSSLSASVGDRVTITCRASENVGNFGISFVNWYQQKPGKAPKLLIYAASNLGSGVPSRFSGSGSGTDFTLTISSLQPEDFATYYCQQSKGVPWTFGQGTKVEIK

SEQ ID NO: 67 - protein, 111 aa, synthetic construct
DIQMTQSPSSLSASVGDRVTITCRASENVGNFGISFVNWFQQKPGKAPKLLIYAASNLGSGDPSRFSGSGSGTDFTLTISSLQPEDFATYFCQQSKGVPWTFGQGTKVEIK

SEQ ID NO: 68 - protein, 111 aa, synthetic construct
DIQLTQSPSSLSASVGDRVTITCRASENVGNFGISFVNWFQQKPGKAPKLLIYAASNLGSGDPARFSGSGSGTDFTLTISSLQPEDFATYFCQQSKGVPWTFGGGTKVEIK

SEQ ID NO: 69 - protein, 111 aa, synthetic construct
DIVLTQSPSSLSASVGDRATITCRASENVGNFGISFVNWFQQKPGKPPKLLIYAASNLGSGDPARFSGSGSGTDFTLTISSLQPEDFATYFCQQSKGVPWTFGGGTKLEIK

SEQ ID NO: 70 - protein, 119 aa, synthetic construct
QVQLVQSGAEVKKPGASVKVSCKASGYTFTNYWMNWVRQAPGQGLEWMGRIDAYAGETHYNQKFKDRVTMTRDTSTSTVYMELSSLRSEDTAVYYCARGYGDAYGMDHWGQGTLVTVSS

SEQ ID NO: 71 - protein, 119 aa, synthetic construct
QVQLVQSGAEVVKPGASVKVSCKASGYTFTNYWMNWVRQAPGQGLEWIGRIDAYAGETHYNQKFKDRVTMTVDTSTSTVYMELSSLRSEDTAVYYCARGYGDAYGMDHWGQGTLVTVSS

SEQ ID NO: 72 - protein, 119 aa, synthetic construct
QVQLVQSGAEVVKPGASVKLSCKASGYTFTNYWMNWVKQAPGQGLEWIGRIDAYAGETHYNQKFKDRATLTVDKSTSTVYMELSSLRSEDTAVYYCARGYGDAYGMDHWGQGTLVTVSS

SEQ ID NO: 73 - protein, 119 aa, synthetic construct
QVQLVQSGAEVVKPGASVKLSCKASGYTFTNYWMNWVKQRPGQGLEWIGRIDAYAGETHYNQKFKDRAILTVDKSTSTAYMELRSLRSEDTAVYYCARGYGDAYGMDHWGQGTLVTVSS

SEQ ID NO: 74 - protein, 111 aa, synthetic construct
DIQMTQSPSSLSASVGDRVTITCRASESVDNYGISFMNWYQQKPGKAPKLLIYAASRQGSGVPSRFSGSGSGTDFTLTISSLQPEDFATYYCQQSKEVPWTFGQGTKVEIK

SEQ ID NO: 75 - protein, 111 aa, synthetic construct
DIQMTQSPSSLSASVGDRVTITCRASESVDNYGISFMNWFQQKPGKAPKLLIYAASRQGSGDPSRFSGSGSGTDFTLTISSLQPEDFATYFCQQSKEVPWTFGQGTKVEIK

SEQ ID NO: 76 - protein, 111 aa, synthetic construct
DIQLTQSPSSLSASVGDRVTITCRASESVDNYGISFMNWFQQKPGKAPKLLIYAASRQGSGDPARFSGSGSGTDFTLTISSLQPEDFATYFCQQSKEVPWTFGGGTKVEIK

SEQ ID NO: 77 - protein, 111 aa, synthetic construct
DIVLTQSPSSLSASVGDRATITCRASESVDNYGISFMNWFQQKPGKPPKLLIYAASRQGSGDPARFSGSGSGTDFTLTISSLQPEDFATYFCQQSKEVPWTFGGGTKLEIK

SEQ ID NO: 78 - protein, 118 aa, synthetic construct
QVQLVQSGAEVKKPGASVKVSCKASGFTFSSYGMSWVRQAPGQGLEWMGTISGGGSYTYYPESVKGRVTMTTDTSTSTAYMELRSLRSDDTAVYYCARRDLRSAMDYWGQGTLVTVSS

SEQ ID NO: 79 - protein, 118 aa, synthetic construct
QVQLVQSGAEVVKPGASVKVSCKASGFTFSSYGMSWVRQAPGQGLEWVGTISGGGSYTYYPESVKGRFTITRDTSTSTAYMELRSLRSDDTAVYYCARRDLRSAMDYWGQGTLVTVSS

SEQ ID NO: 80 - protein, 118 aa, synthetic construct
QVQLVQSGAEVVKPGASVKLSCKASGFTFSSYGMSWVRQAPGQGLEWVATISGGGSYTYYPESVKGRFTITRDNSTSTLYMEMRSLRSDDTAVYYCARRDLRSAMDYWGQGTLVTVSS

SEQ ID NO: 81 - protein, 118 aa, synthetic construct
QVKLVESGAEVVKPGASVKLSCKASGFTFSSYGMSWVRQAPGQRLEWVATISGGGSYTYYPESVKGRFTITRDNSTSTLYLEMRSLRSDDTAVYYCARRDLRSAMDYWGQGTLVTVSS

SEQ ID NO: 82 - protein, 107 aa, synthetic construct
DVVMTQSPLSLPVTLGQPASISCKASQNVDINVAWFQQRPGQSPRRLIYSASYRYSGVPDRFSGSGSGTDFTLKISRVEAEDVGVYYCQQYNAYPLTFGQGTKLEIK

SEQ ID NO: 83 - protein, 107 aa, synthetic construct
DIVMTQSPLSLPVTLGQPASISCKASQNVDINVAWYQQRPGQSPRALIYSASYRYSGVPDRFSGSGSGTDFTLKISRVEAEDVGVFYCQQYNAYPLTFGQGTKLEIK

SEQ ID NO: 84 - protein, 107 aa, synthetic construct
DIVMTQSPLSLPTTLGQPASISCKASQNVDINVAWYQQRPGQSPRALIYSASYRYSGVPDRFSGSGSGTDFTLTISRVEAEDVGVFYCQQYNAYPLTFGGGTKLEIK

SEQ ID NO: 85 - protein, 107 aa, synthetic construct
DIVMTQSPLSLPTTLGQPVSVSCKASQNVDINVAWYQQRPGQSPRALIYSASYRYSGVPDRFSGSGSGTDFTLTISRVEAEDVAVFFCQQYNAYPLTFGGGTKLEIK